(19) United States
(12) Patent Application Publication
(10) Pub. No.: US 2005/0260699 A1
(43) Pub. Date: Nov. 24, 2005

(54) Carotenoid biosynthesis

(76) Inventors: Mervyn L. Desouza, Plymouth, MN (US); Holly Jessen, Chanhassen, MN (US); William A. Schroeder, Brooklyn Park, MN (US); Ravi R. Gokarn, Plymouth, MN (US)

Correspondence Address: Paula A Degrandis;Cargill Incorporated, PO Box 5624, Minneapolis, MN, 55440-5624, US

(21) Appl. No.: 10/432,483

(22) PCT Filed: Nov. 21, 2001
(86) PCT No.: PCT/US01/43906

§ 371 Date: May 22, 2003

Related U.S. Application Data
(60) Provisional application No. 60/252,749, filed on Nov. 22, 2000.

(51) Int. Cl. C12P 23/00

(52) U.S. Cl. 435/67

(57) ABSTRACT

The invention provides materials and methods that can be used to make carotenoids having greater than 40 carbon atoms (C>40). The invention also provides isolated nucleic acid molecules that encode polypeptides that allow C40 carotenoids to be converted to C50 carotenoids. The isolated nucleic acid molecules can be introduced into production cells, wherein the production cell becomes capable of the biosynthesis and the conversion of the C>40 carotenoids.

### FIELD OF THE INVENTION

This invention relates to materials and methods for making carotenoids.

### BACKGROUND

Carotenoids have significant utility in pigment and anti-oxidant applications. For example, many of the red, yellow, and orange colors observed in nature are pigments provided by one or more carotenoids. Carotenoids are among the best antioxidants provided by nature—orders of magnitude better than other naturally available materials such as vitamin C or vitamin E. The carotenoid molecule comprises multiples of the isoprene molecule, a C5 hydrocarbon with two double bonds. In view of the dual unsaturation of the isoprene molecule, the class of carotenoid molecules is characterized by long organic chains with conjugated double bonds. It has been shown that the high antioxidant capacity and the vivid pigmentation are directly attributable to the long chains of conjugated double bonds. For example, Conn et al. J. Photochemistry Photobiology B, 11: 41-47, 1991 compared the common β-carotene—a C40 carotenoid having 11 conjugated double bonds—with a chemically synthesized C50 β-carotene having 15 conjugated double bonds and with a chemically synthesized C60 β-carotene having 19 conjugated double bonds. The Conn et al. study concluded, based on quenching of singlet oxygen, that the efficiency of antioxidant activity increased with increasing numbers of conjugated double bonds.

The literature is replete with details concerning the biosynthesis of C40 carotenoids, including details concerning the associated genes and the enzymes encoded by the genes. However, the biosynthesis and biochemical properties of C>40 carotenoids is poorly understood relative to the level of knowledge of C40 carotenoids. Ironically, C>40 carotenoids have the potential to be more effective antioxidants, to provide greater health benefits, and to generate novel improved colored pigments (i.e. pigments of longer wavelength absorbance maxima).

There are numerous reports in the literature of bacteria that are capable of producing C50 carotenoids. Examples of such bacteria include Halobacterium salinarium, Cellulomonas biazotea, Arthrobacter glacialis, Corynebacterium poinsettias, Micrococcus luteus, and Agromyces mediolanus. Examples of C50 carotenoids produced by Micrococcus luteus, Agromyces mediolanus, and Halobacterium salinarium are shown in FIG 11.

Three C50 carotenoids (molecular formulae C₅₀H₇₂O₂) have been isolated from the psychrophilic bacterium Arthrobacter glacialis, including bicyclic decaprenoxanthin, aliphatic bisanhydrobacterioruberin, and monocyclic A.g. 470 (Arpin N, et al. Acta Chem Scand B 29:921-6, 1975).

It is clear that carotenoid characteristics such as antioxidant and pigment capabilities improve with a greater number of conjugated double bonds. In view of production and other technical limitations, however, commercial use of carotenoids has been substantially limited to those no longer than C40. To allow sufficient production of the C50 carotenoid to commercially utilize its improved properties, it would be desirable to have the capability to convert C40 carotenoids to C50 carotenoids by genetic manipulation.

### SUMMARY OF THE INVENTION

The present invention is based on isolated nucleic acid molecules that encode polypeptides that allow C40 carotenoids to be converted to carotenoids having greater than 40 carbon atoms (C>40), such as a C50 carotenoid. These polypeptides can be used in vitro or in vivo. The isolated nucleic acid molecules can be introduced into a production cell, wherein the production cell becomes capable of converting a C40 carotenoid to a C>40 carotenoid, such as a C50 carotenoid.

In one aspect, the invention features an isolated polypeptide, isolated nucleic acid molecules encoding the polypeptide, and production cells that include the isolated nucleic acid molecules. The isolated polypeptide includes at least one amino acid sequence selected from the group consisting of (a) the amino acid sequence set forth in SEQ ID NOS: 04, 05, 06, 10, 11, 12, 17, 18, 19, 20, 24, 25 or 26; (b) an amino acid sequence having at least 10 contiguous amino acid residues of the amino acid sequence set forth in SEQ ID NOS: 04, 05, 06, 10, 11, 12, 17, 18, 19, 20, 24, 25 or 26; (c) an amino acid sequence having one or more conservative amino acid substitutions within the amino acid sequence set forth in SEQ ID NOS: 04, 05, 06, 10, 11, 12, 17, 18, 19, 20, 24, 25 or 26; and (d) an amino acid sequence having at least 65% sequence identity with the amino acid sequences of (a) or (b). Polypeptides at least 10 amino acid residues in length are useful for, among other things, generating specific binding agents, such as antibodies. Polypeptides having at least 65% sequence identity with the amino acid sequences of (a) or (b) are useful for creating specific binding agents that vary in binding strength, as well as for creating polypeptides with enzymatic activities that vary in binding strength (Km) and/or turnover rate (Kcat).

The nucleic acid molecule can encode a polypeptide capable of converting a C40 carotenoid to a C50 carotenoid, a C40 carotenoid to a C45 carotenoid, a C45 carotenoid to a C50 carotenoid, or capable of synthesizing a C40 carotenoid. These polypeptides can be used in vitro or in vivo.

The invention also features an isolated nucleic acid molecule or a production cell containing the nucleic acid molecule. The nucleic acid molecule includes a nucleic acid sequence selected from the group consisting of: (a) the nucleotide sequence set forth in SEQ ID NOS: 01, 02, 03, 07, 08, 09, 13, 14, 15, 16, 21, 22 or 23; (b) a nucleic acid sequence having at least 10 contiguous nucleotides of the nucleotide sequence set forth in SEQ ID NOS: 01, 02, 03, 07, 08, 09, 13, 14, 15, 16, 21, 22 or 23; (c) a nucleic acid sequence that hybridizes under moderately stringent conditions to the nucleotide sequence of (a); and (d) a nucleic acid sequence having 65% sequence identity with the nucleic acid sequence of (a) or (b). These nucleic acid molecules are useful for identifying other nucleic acid sequences that encode polypeptides with similar enzymatic activities to those described herein. Methods such as the polymerase chain reaction (PCR), which utilizes short fragments of the disclosed sequences, or Northern and/or Southern blotting procedures which utilize slightly longer fragments, can be used to identify substantially similar sequences.

In another aspect, the invention features a method for making a C50 carotenoid. The method includes contacting at least one of the polypeptides described above with a C40 carotenoid such that the C50 carotenoid is made. A C50 carotenoid also can be made by culturing the production cell described above under conditions wherein the C50 carotenoid is made.

In yet another aspect, the invention features a method for making a C45 carotenoid. The method includes contacting at least one of the polypeptides described above with a C40 carotenoid such that the C45 carotenoid is made. A C45 carotenoid also can be made by culturing the production cell described above under conditions wherein the C45 carotenoid is made.

The invention also features a method for making a polypeptide. The method includes culturing the production cell described above under conditions such that the polypeptide is made.

In another aspect, the invention features a specific binding agent that binds to the polypeptide described above.

In yet another aspect, the invention features a method for making a C>40 carotenoid. The method includes culturing a production cell, wherein the production cell includes an exogenous nucleic acid molecule, wherein the exogenous nucleic acid molecule encodes a polypeptide that elongates a C>40 carotenoid by at least one carbon atom, wherein the product produced by the polypeptide is a carotenoid having a carbon backbone of >40 carbon atoms. The use of the term carbon backbone refers to the single contiguous chain of carbon-carbon bonds that are found in carotenoids. The exogenous nucleic acid molecule can include a nucleic acid sequence selected from the group consisting of: (a) the nucleotide sequence set forth in SEQ ID NOS: 01, 02, 03, 07, 08, 09, 13, 14, 15, 16, 21, 22 or 23; (b) a nucleotide sequence having at least 10 consecutive nucleotides of the nucleotide sequence set forth in SEQ ID NOS: 01, 02, 03, 07, 08, 09, 13, 14, 15, 16, 21, 22 or 23; (c) a nucleic acid sequence that hybridizes under moderately stringent conditions to the nucleotide sequence of (a); and (d) a nucleic acid sequence having 65% sequence identity with the nucleic acid sequence of (a) or (b). The exogenous nucleic acid molecule can encode a polypeptide, wherein the polypeptide includes an amino acid sequence selected from the group consisting of: (a) the amino acid sequence of SEQ ID NOS: 04, 05, 06, 10, 11, 12, 17, 18, 19, 20, 24, 25 or 26; (b) an amino acid sequence having at least 10 contiguous amino acid residues of the amino acid sequence set forth in SEQ ID NOS: 04, 05, 06, 10, 11, 12, 17, 18, 19, 20, 24, 25 or 26; (c) an amino acid sequence having one or more conservative amino acid substitutions within the amino acid sequence of SEQ ID NOS: 04, 05, 06, 10, 11, 12, 17, 18, 19, 20, 24, 25 or 26; and (d) an amino acid sequence having at least 65% sequence identity with the amino acid sequences of (a) or (b).

These and other aspects of the invention will are discussed in more detail in the following detailed description.

Sequence Listing

The nucleic and amino acid sequences listed in the accompanying sequence listing are shown using standard letter abbreviations for nucleotide bases, and three-letter codes for amino acids. Only one strand of each nucleic acid sequence is shown, but the complementary strand is understood to be included by any reference to the displayed strand.

SEQ ID NO: 01 is the nucleic acid sequence for the A. mediolanus lctA gene (a lycopene cyclase).

SEQ ID NO: 02 is the nucleic acid sequence for the A. mediolanus lctB gene.

SEQ ID NO: 03 is the nucleic acid sequence for the A. mediolanus lctC gene.

SEQ ID NO: 04 is the amino acid sequence encoded by SEQ ID NO: 01.

SEQ ID NO: 05 is the amino acid sequence encoded by SEQ ID NO: 02.

SEQ ID NO: 06 is the amino acid sequence encoded by SEQ ID NO: 03.

SEQ ID NO: 07 is the nucleic acid sequence for the M. luteus lctA gene.

SEQ ID NO: 08 is the nucleic acid sequence for the M. luteus lctB gene.

SEQ ID NO: 09 is the nucleic acid sequence for the M. luteus lctC gene.

SEQ ID NO: 10 is the amino acid sequence encoded by SEQ ID NO: 07.

SEQ ID NO: 11 is the amino acid sequence encoded by SEQ ID NO: 08.

SEQ ID NO: 12 is the amino acid sequence encoded by SEQ ID NO: 09.

SEQ ID NO: 13 is the nucleic acid sequence for the A. mediolanus idi gene.

SEQ ID NO: 14 is the nucleic acid sequence for the A. mediolanus crtE gene.

SEQ ID NO: 15 is the nucleic acid sequence for the A. mediolanus crtB gene.

SEQ ID NO: 16 is the nucleic acid sequence for the A. mediolanus crtI gene.

SEQ ID NO: 17 is the amino acid sequence encoded by SEQ ID NO: 13.

SEQ ID NO: 18 is the amino acid sequence encoded by SEQ ID NO: 14.

SEQ ID NO: 19 is the amino acid sequence encoded by SEQ ID NO: 15.

SEQ ID NO: 20 is the amino acid sequence encoded by SEQ ID NO: 16.

SEQ ID NO: 21 is the nucleic acid sequence for the M. lueus crtE gene.

SEQ ID NO: 22 is the nucleic acid sequence for the M. lueus crtB gene.

SEQ ID NO: 23 is the nucleic acid sequence for the M. lueus crtI gene.

SEQ ID NO: 24 is the amino acid sequence encoded by SEQ ID NO: 21.

SEQ ID NO: 25 is the amino acid sequence encoded by SEQ ID NO: 22.

SEQ ID NO: 26 is the amino acid sequence encoded by SEQ ID NO: 23.

SEQ ID NOS: 27-30 are primers used to amplify regions of the carotenogenic operon from the Y1 clone.

SEQ ID NOS: 31 and 32 are primers used to amplify ORFY.

SEQ ID NO: 33 is a primer used in combination with SEQ ID NO: 32, to amplify the region of A. mediolanus genomic DNA containing the X1, X2, and Y ORFs.

SEQ ID NOS: 34 and 35 are primers used to amplify a mutated ORFX1, ORFX2, and ORFY fragment.

SEQ ID NOS: 36 and 37 are primers used to amplify a mutated ORFX2 fragment.

SEQ ID NOS: 38 and 39 are primers used to amplify a mutated ORFY fragment.

SEQ ID NOS: 40 and 41 are primers used to make a probe to identify M. lueus homologs.

SEQ ID NOS: 42-45 are primers used for M. lueus genomic walking.

### BRIEF DESCRIPTION OF THE DRAWINGS

FIG. 1 is the nucleotide sequence of the 9-Kb Y1 operon—the C50 carotenoid producing operon from A. mediolanus.

FIG. 2 contains HPLC chromatograms of carotenoid extracts from A. mediolanus, E. coli formed with the idi-Y construct, E. coli transformed with the idi-crtI construct, a lycopene standard, and E. coli transformed with the idi-X2 construct.

FIG. 3A contains chromatograms of carotenoid extracts from A. mediolanus and E. coli transformed with the idi-ORFY construct (Yellow E. coli clone Y33). The two analyses show a peak at virally the same retention time.

FIG. 3B contains visible spectra for the A. mediolanus extract and an extract from E. coli transformed with the idi-ORFY (Yellow E. coli clone Y33). The visible spectra for both peaks are virtually identical.

FIG. 4 is mass spectra of carotenoid extracts from A. mediolanus and from E. coli transformed with the idi-ORFY construct (Yellow E. coli clone Y33). The analysis confirmed that the compound from clone Y33 and A. mediolanus at a retention time of 7 minutes had the same mass.

FIG. 5 contains HPLC chromatograms of carotenoids extracted from E. coli transformed with the idi-crtI construct and a lycopene standard (Sigma).

FIG. 6 contains visible spectra for carotenoids extracted from E. coli transformed with the idi-crtI construct and a lycopene standard (Sigma). The visible spectra are virtually identical.

FIG. 7 contains mass spectra of a lycopene standard, carotenoids produced in E. coli transformed with the idi-crtI construct and carotenoids produced in E. coli transformed with the idi-ORFX2 construct.

FIG. 8 is a visible-spectrophotometric analysis of carotenoid extracts from A. mediolanus and mutant E. coli clones. The mutant E. coli clones produced the C40 carotenoid lycopene and no C50 carotenoid, while A. mediolanus produced the C50 carotenoid decaprenoxanthin.

FIG. 9 is a schematic of the arrangement of genes within the biosynthetic pathway for the production of a C50 carotenoid for A. mediolanus, M. lueus, C. glutamicum, H. salinarium, and M. thermoautotrophicum.

FIG. 10 is a schematic of the biosynthetic pathway for the production of decapremioxan in A. mediolanus and the postulated role of the lctA, lctB, and lctC genes.

FIG. 11 depicts examples of C50 carotenoid structures reported in the literature.

FIG. 12 is the nucleotide sequence of the C50-carotenoid producing operon from M. luteus ATCC 383.

### DETAILED DESCRIPTION

I. Terms

Unless otherwise noted, technical terms are used according to conventional usage. Definitions of common terms in molecular biology may be found in Benjamin Lewin, Genes VII, Oxford University Press, 1999 (ISBN β-19-879276-X); Kendrew et. al. (editors), The Encyclopedia of Molecular Biology, Blackwell Science Ltd., 1994 (ISBN 0-632-021182-9); and Robert A. Meyers (editor), Molecular Biology and Biotechnology; a Comprehensive Desk Reference, BCH Publishers, Inc., 1995 (ISBN 1-56081-569-8).

Carotenoid—A molecule that includes at least two isoprenoid units joined in such a manner that the two joined isoprenoid units have two methyl groups in a 1,6-positional relationship. The term “carotenoid” also includes derivatives having one or more hydrogen atoms replaced with a substituent group or atom. Non-limiting examples of substituents include 1) hydroxyl groups (yielding an alcohol); 2) methoxyl groups (derived from an alcohol); 3) glycosyl (sugar) residues (attached by an ether bond); 4) fatty acid residues (attached by an ester bond); 5) carbonyl groups (yielding aldehydes or ketones); 6) sulfates; 7) carboxylic acids; and 8) epoxides. Additional carbon atoms can be added via the substituent group. Hydrogen atoms can be replaced anywhere on the molecule, including within the methyl groups in the 1-6 positional relationship. Non-limiting examples of typical carotenoids include β-carotene, phytoene, lycopene, dehydrogenans P-452, decaprenoxanthin, 4,4′-diapophytoene, and norbixin.

CX—The carotenoid molecules of the present application are characterized by the term “CX”, wherein “C” refers to carbon atoms and the “X” refers to the total number of carbon atoms in the isoprenoid units of the carotenoid molecule.

C>X—The designation “C>X carotenoid” means a carotenoid having more than X carbon atoms total in the isoprenoid units of the carotenoid molecule. Similarly C<X is used to identify a carotenoid having less than X carbon atoms.

Homology—A term referring to the sequence identity between two or more sequences.

Isoprenoid—A molecule that is a multiple of the C5 hydrocarbon isoprene (2-methyl-1,2-butadiene).

Polypeptide—The term “polypeptide” includes any chain of amino acids at least eight amino acids in length, regardless of post-translational modification.

Nucleic acid—The term “nucleic acid” as used herein encompasses both RNA and DNA including, without limitation, cDNA, genonic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

Isolated—The term “isolated” as used herein with reference to a polypeptide refers to a polypeptide that has been separated from the cellular components that naturally accompany it. Typically, the polypeptide is isolated when it is at least 60% (e.g., 70%, 80%, 90%, 92%, 95%, 98%, or 99%), by weight, free from proteins and naturally-occurring organic molecules that are naturally associated with it. In general, an isolated polypeptide will yield a single major band on a non-reducing polyacrylamide gel.

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurning nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

Exogenous: The term “exogenous” as used herein with reference to nucleic acid and a particular cell refers to any nucleic acid that does not originate from that particular cell as found in nature. Thus, non-naturally-occurring nucleic acid is considered to be exogenous to a cell once introduced into the cell. Nucleic acid that is naturally-occurring also can be exogenous to a particular cell. For example, an entire chromosome isolated from a cell of person X is an exogenous nucleic acid with respect to a cell of person Y once that chromosome is introduced into Y's cell.

ORF (open reading frame)—An “ORF” is a series of nucleotide triplets (codons) encoding a sequence of amino acids at least 100 amino acids in length without any termination codons.

Probes and primers—Nucleic acid probes and primers may be prepared readily based on the amino acid sequences and nucleic acid sequences provided by this invention.

A “probe” comprises an isolated nucleic acid attached to a detectable label or reporter molecule. Typical labels include radioactive isotopes, ligands, chemiluminescent agents, and polypeptides. Methods for labeling and guidance in the choice of labels appropriate for various purposes are discussed in, e.g., Sambrook et al. (ed.), Molecular Cloning: A Laboratory Manual 2nd ed., vol. 1-3, Cold Spring Harbor Laboratory Press, Cold Spring Harbor, N.Y., 1989, and Ausubel et a. (ed.) Current Protocols in Molecular Biology Greene Publishing and Wiley-Interscience, New York (with periodic updates), 1987.

“Primers” are short nucleic acids, preferably DNA oligonucleotides, 10 nucleotides or more in length. A primer may be annealed to a complementary target DNA strand by nucleic acid hybridization to form a hybrid between the primer and the target DNA strand, and then extended along the target DNA strand by a DNA polymerase. Primer pairs can be used for amplification of a nucleic acid sequence, e.g., by the polymerase chain reaction (PCR), or other nucleic-acid amplification methods known in the art.

Methods for preparing and using probes and primers are described, for example, in references such as Sambrook et al. (ed.), Molecular Cloning: A Laboratory Manual, 2nd ed., vol. 1-3, Cold Spring Harbor Laboratory Press, Cold Spring Harbor, N.Y., 1989; Ausubel et al. (ed.), Current Protocols in Molecular Biology, Greene Publishing and Wiley-Interscience, New York (with periodic updates), 1987; and Innis et al., PCR Protocols: A Guide to Methods and Aplications, Academic Press: San Diego, 1990. PCR primer pairs can be derived from a known sequence, for example, by using computer programs intended for that purpose such as Primer Designer 3 for Windows by Scientific & Educational Software (Durham, N.C.).

One of skill in the art will appreciate that the specificity of a particular probe or primer generally increases with the length of the probe or primer. Thus, for example, a primer comprising 20 consecutive nucleotides will anneal to a target having a higher specificity than a corresponding primer of only 15 nucleotides. Thus, in order to obtain greater specificity, probes and primers may be selected that comprise, for example, 10, 20, 25, 30, 35, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700 or more consecutive nucleotides.

Recombinant—A “recombinant” nucleic acid is one having (1) a sequence that is not naturally occurring in the organism in which it is expressed or (2) a sequence made by an artificial combination of two otherwise-separated, shorter sequences. This artificial combination is often accomplished by chemical synthesis or, more commonly, by the artificial manipulation of isolated segments of nucleic acids, e.g., by genetic engineering techniques. “Recombinant” is also used to describe nucleic acid molecules that have been artificially manipulated, but contain the same regulatory sequences and coding regions that are found in the organism from which the nucleic acid was isolated.

Sequence identity—The similarity between two or more nucleic acid sequences or amino acid sequences is referred to as “Sequence Identity.” The “percent sequence identity” between a particular nucleic acid or amino acid sequence and a sequence referenced by a particular sequence identification number is determined as follows.

First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained at www.fr.com or www.ncbi.nlm.nih.gov. Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1;-r is set to 2; and all other options are left at their default setting. For example, the following command can be used to generate an output file containing a comparison between two sequences: C:\B12seq -i c:\seq1.txt -j c:\seq2.txt -p blastn -o c:\output.txt -q −1-r 2.

To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seq1.txt -j c:\seq2.txt-p blastp -o c:\output.txt.

If the target sequence shares homology with any portion of the identified sequence (i.e., the sequence identified by a SEQ ID NO herein), then the designated output file will present those regions of homology as aligned sequences. If the target sequence does not share homology with any portion of the identified sequence, then the designated output file will not present aligned sequences. Once aligned, a length is determined by counting the number of consecutive nucleotides or amino acid residues from the target sequence presented in alignment with sequence from the identified sequence starting with any matched position and ending with any other matched position. A matched position is any position where an identical nucleotide or amino acid residue is presented in both the target and identified sequence. Gaps presented in the target sequence are not counted since gaps are not nucleotides or amino acid residues. Likewise, gaps presented in the identified sequence are not counted since target sequence nucleotides or amino acid residues are counted, not nucleotides or amino acid residues from the identified sequence.

The percent identity over a determined length is determined by counting the number of matched positions over that length and dividing that number by the length followed by multiplying the resulting value by 100. For example, if (1) a 1000 nucleotide target sequence is compared to the sequence set forth in SEQ ID NO: 1, (2) the B12seq program presents 200 nucleotides from the target sequence aligned with a region of the sequence set forth in SEQ ID NO: 1 where the first and last nucleotides of that 200 nucleotide region are matches, and (3) the number of matches over those 200 aligned nucleotides is 180, then the 1000 nucleotide target sequence contains a length of 200 and a percent identity over that length of 90 (i.e., 180/200*100=90).

It will be appreciated that a single nucleic acid or amino acid target sequence that aligns with an identified sequence can have many different lengths with each length having its own percent identity. For example, a target sequence containing a 20-nucleotide region (SEQ ID NO: 46) that aligns with an identified sequence (SEQ ID NO: 47) as follows has many different lengths including those listed in Table 1.

| | |
|---|---|
| | 1 20 |
| Target Sequence: | AGGTCGTGTACTGTCAGTCA |
| | \| \|\| \|\|\| \|\|\|\| \|\|\|\| \| |
| Identified Sequence: | ACGTGGTGAACTGCCAGTGA |

TABLE 1

| Starting | Ending Posi- | | | |
|---|---|---|---|---|
| position | tion | Length | Matched Positions | Percent Identity |
| 1 | 20 | 20 | 15 | 75.0 |
| 1 | 18 | 18 | 14 | 77.8 |
| 1 | 15 | 15 | 11 | 73.3 |
| 6 | 20 | 15 | 12 | 80.0 |
| 6 | 17 | 12 | 10 | 83.3 |
| 6 | 15 | 10 | 8 | 80.0 |
| 8 | 20 | 13 | 10 | 76.9 |
| 8 | 16 | 9 | 7 | 77.8 |

It is noted that the percent identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It is also noted that the length value will always be an integer.

Accordingly, the invention provides nucleic acid sequences and amino acid sequences that share at least 60, 65, 70, 75, 80, 85, 90, 95, 97, and 98% sequence identity to SEQ ID NOS: 01, 02, 03, 07, 08, 09, 13, 14, 15, 16, 21, 22, and 23, and SEQ ID NOS: 04, 05, 06, 10, 11, 12, 17, 18, 19, 20, 24, 25, and 26, respectively.

Specific binding agent—A “specific binding agent” is an agent that is capable of specifically binding to the polypeptides of the present invention, and may include polyclonal antibodies, monoclonal antibodies (including humanized monoclonal antibodies) and fragments of monoclonal antibodies such as Fab, F(ab′)2 and Fv fragments, as well as any other agent capable of specifically binding to the epitopes on the proteins.

Antibodies to the polypeptides, and fragments thereof, of the present invention may be useful for purification of the polypeptides. The amino acid and nucleic acid sequences provided herein allow for the production of specific antibody-based binding agents to these polypeptides.

Monoclonal or polyclonal antibodies may be produced to full-length polypeptides, polypeptides that are less than full-length, or variants thereof. Optimally, antibodies raised against epitopes on these antigens will specifically detect the polypeptides. That is, antibodies raised against the polypeptide would recognize and bind the polypeptides, and would not substantially recognize or bind to other polypeptides. The determination that an antibody specifically binds to an antigen is made by any one of a number of standard immunoassay methods; for instance, Western blotting, Sambrook et al. (ed.), Molecular Cloning: A Laboratory Manual, 2nd ed., vol. 1-3, Cold Spring Harbor Laboratory Press, Cold Spring Harbor, N.Y., 1989.

To determine that a given antibody preparation (such as a preparation produced in a mouse against SEQ ID NO: 4) specifically detects a polypeptide having the amino acid sequence of SEQ ID NO: 4 by Western blotting, total cellular protein is extracted from cells and electrophoresed through a sodium dodecyl sulfate (SDS) polyacrylamide gel. The proteins are then transferred to a membrane (for example, nitrocellulose) and the antibody preparation is incubated with the membrane. After washing the membrane to remove non-specifically bound antibodies, the presence of specifically bound antibodies can be detected with anti-mouse antibody conjugated to an enzyme such as alkaline phosphatase; application of 5-bromo-4-chloro-3-indolyl phosphate/nitro blue tetrazolium results in the production of a densely blue-colored compound by immuno-localized alkaline phosphatase.

Isolated polypeptides suitable for use as an immunogen can be isolated from transfected cells, transformed cells, or from wild-type cells. Concentration of protein in the final preparation is adjusted, for example, by concentration on an Amicon filter device, to the level of a few micrograms per milliliter. Polypeptides that range in size from eight amino acid residues to a full-length polypeptide having enzymatic activity can be utilized as an immunogen. Polypeptides that are less than full-length may be chemically synthesized using standard methods, or may be obtained by cleavage of the whole polypeptide followed by purification of the desired size of polypeptide. Polypeptides as short as eight amino acids in length are immunogenic when presented to an immune system in the context of a Major Histocompatibility Complex (MHC) molecule, such as MHC class I or MHC class II. Accordingly, polypeptides comprising at least 8, 10, 20, 25, 30, 35, 40, 45, 50, 55, 60, 70, 80, 90, 100, 150, 200, 250, 300, 350, 400, 450, 500, 550, 600, 650, 700, 750, 800, 900, 1000, 1050, 1100, 1150, 1200, 1250, 1300, 1350 or more consecutive (contiguous) amino acids of the disclosed amino acid sequences may be employed as immunogens for producing antibodies.

Monoclonal antibodies to any of the polypeptides disclosed herein can be prepared from murine hybridomas according to the classic method of Kohler & Milstein (Nature 256:495 (1975)) or a derivative method thereof.

Polyclonal antiserum containing antibodies to the heterogeneous epitopes of any polypeptide disclosed herein can be prepared by immunizing suitable animals with a polypeptide, which can be unmodified or modified to enhance immunogenicity. An effective immunization protocol for rabbits can be found in Vaitukaitis et al. (J. Clin. Endocrinol. Metab. 33:988-991 (1971)).

Antibody fragments can be used in place of whole antibodies and can be readily expressed in prokaryotic host cells. Methods of making and using immunologically effective portions of monoclonal antibodies, also referred to as “antibody fragments,” are well known and include those described in Better & Horowitz (Methods Enzymol. 178:476-496 (1989)), Glockshuber et al. (Biochemistry 29:1362-1367 (1990), U.S. Pat. No. 5,648,237 (“Expression of Functional Antibody Fragments”), U.S. Pat. No. 4,946,778 (“Single Polypeptide Chain Binding Molecules”), U.S. Pat. No. 5,455,030 (“Immunotherapy Using Single Chain Polypeptide Binding Molecules”), and references cited therein.

Hybridization—“Hybridization” is a method of testing for complementarity in the base sequence of two nucleic acid molecules from different sources, and is based on the ability of complementary single-stranded DNA and/or RNA molecules to form a duplex molecule. Nucleic acid hybridization techniques can be used to obtain an isolated nucleic acid within the scope of the invention. Briefly, any nucleic acid having homology to a sequence set forth in SEQ ID NOS: 01, 02, 03, 07, 08, 09, 13, 14, 15, 16, 21, 22, and 23 can be used as a probe to identify a similar nucleic acid by hybridization under conditions of moderate to high stringency. Once identified, the nucleic acid then can be purified, sequenced, and analyzed to determine whether it is within the scope of the invention as described herein.

Hybridization can be done by Southern or Northern analysis to identify a DNA or RNA sequence, respectively, that hybridizes with a nucleic acid of the invention (e.g., a probe). The probe can be labeled with a biotin, digoxygenin, an enzyme, or a radioisotope such as ³²P. The DNA or RNA to be analyzed can be electrophoretically separated on an agarose or polyacrylamide gel, transferred to nitrocellulose, nylon, or other suitable membrane, and hybridized with the probe using standard techniques well known in the art such as those described in sections 7.39-7.52 of Sambrook et al., (1989) Molecular Cloning, second edition, Cold Spring Harbor Laboratory, Plainview, N.Y. Typically, a probe is at least about 20 nucleotides in length. For example, a probe corresponding to a 20 nucleotide sequence set forth in SEQ ID NO: 01, 02, 03, 07, 08, 09, 13, 14, 15, 16, 21, 22, and 23 can be used to identify an identical or similar nucleic acid. In addition, probes longer or shorter than 20 nucleotides can be used.

The invention also provides isolated nucleic acid molecules that are at least about 12 bases in length (e.g., at least about 13, 14, 15, 16, 17, 18, 19, 20, 25, 30, 40, 50, 60, 100, 250, 500, 750, 1000, 1500, 2000, 3000, 4000, or 5000 bases in length) and that hybridize, under moderate to highly stringent hybridization conditions, to the sense or antisense strand of a nucleic acid having the sequence set forth in SEQ ID NO: 01, 02, 03, 07, 08, 09, 13, 14, 15, 16, 21, 22, or 23.

For the purpose of this invention, moderately stringent hybridization conditions mean the hybridization is performed at about 42° C. in a hybridization solution containing 25 mM KPO₄ (pH 7.4), 5× SSC, 5× Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 50° C. with a wash solution containing 2× SSC and 0.1% sodium dodecyl sulfate.

Highly stringent hybridization conditions mean the hybridization is performed at about 42° C. in a hybridization solution containing 25 mM KPO₄ (pH 7.4), 5× SSC, 5× Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 65° C. with a wash solution containing 0.2× SSC and 0.1% sodium dodecyl sulfate.

Sequence Variants—With the provision of the amino acid sequences set forth in SEQ ID NOS: 04, 05, 06, 10, 11, 12, 17, 18, 19, 20, 24, 25, and 26 and the corresponding nucleic acid sequences set forth in SEQ ID NOS: 01, 02, 03, 07, 08, 09, 13, 14, 15, 16, 21, 22, and 23, variants of these sequences can be created. The sequence of these variants share from about 50% to about 99% sequence identity with the corresponding sequence provided in the accompanying sequence listing. In other embodiments, the variants share at least 55, 60, 65, 70, 75, 80, 85, 87, 90, 92, 94, 96, or 98% sequence identity with the sequences described herein.

Variant polypeptides sequences include polypeptides that differ in amino acid sequence from the polypeptides sequences disclosed, but that retain biological activity (e.g., enzymatic activity). Such polypeptides may be produced by manipulating the nucleotide sequence encoding the enzyme using standard procedures such as site-directed mutagenesis or the polymerase chain reaction. The simplest modifications involve the substitution of one or more amino acids for amino acids having similar biochemical properties. These so-called “conservative substitutions” are likely to have minimal impact on the activity of the resultant polypeptide. Table 2 provides examples of conservative substitutions.

TABLE 2

| Original Residue | Conservative Substitution(s) |
|---|---|
| Arg | Lys |
| Asn | Gln |
| Asp | Glu |
| Cys | Ser |
| Gln | Asn |
| Glu | Asp |
| His | Asn; Gln |
| Ile | Leu; Val |
| Leu | Ile; Val |
| Lys | Arg; Gln; His |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

More substantial changes in enzymatic function or other features may be obtained by selecting substitutions that are less conservative than those in Table 2, i.e., selecting residues that differ more significantly in their effect on maintaining: (a) the structure of the polypeptide backbone in the area of the substitution, for example, as a sheet or helical conformation; (b) the charge or hydrophobicity of the molecule at the target site; or (c) the bulk of the side chain. The substitutions that in general are expected to produce the greatest changes in protein properties will be those in which: (a) a hydrophilic residue, e.g., serine or threonine, is substituted for a hydrophobic residue, e.g., leucine, isoleucine, phenylalanine, valine or alanine, or vice versa; (b) a cysteine or proline is substituted for any other residue; (c) a residue having an electropositive side chain, e.g., lysine, arginine, or histidine, is substituted for an electronegative residue, e.g., glutamine or aspartarine, or vice versa; or (d) a residue having a bulky side chain, e.g., phenylalanine, is substituted for one not having a side chain, e.g., glycine, or vice versa. The effects of these amino acid substitutions, deletions, or additions can be assessed for polypeptides having enzyme activity by analyzing the ability of the polypeptide to catalyze the conversion of the same substrate as the related native polypeptide to the same product as the related native polypeptide. Accordingly, polypeptide having 5, 10, 20, 30, 40, 50 or less conservative amino acid substitutions are provided by the invention.

Polypeptides and nucleic acids encoding polypeptides can be produced by standard DNA mutagenesis techniques, for example, M13 primer mutagenesis. Details of these techniques are provided in Sambrook et al. (ed.), Molecular Cloning: A Laboratory Manual 2nd ed., vol. 1-3, Cold Spring Harbor Laboratory Press, Cold Spring, Harbor, N.Y., 1989, Ch. 15. By the use of such techniques, variants may be created that differ in minor ways from the native sequence, yet that still encode a polypeptide having enzymatic activity. In their simplest form, such variants may differ from the disclosed sequences by alteration of the coding region to fit the codon usage bias of the particular organism into which the molecule is to be introduced.

Alternatively, the coding region may be altered by taking advantage of the degeneracy of the genetic code to alter the coding sequence in such a way that, while the nucleotide sequence is substantially altered, it nevertheless encodes a protein having, an amino acid sequence identical or substantially similar to the disclosed polypeptide sequences. For example, the 5th amino acid residue of the SEQ ID NO: 18 is alanine. This is encoded in the open reading frame (ORF) by the nucleotide codon triplet GCG. Because of the degeneracy of the genetic code, three other nucleotide codon triplets—GCA, GCC, and GCT—also code for alanine. Thus, the nucleotide sequence of the ORF can be changed at this position to any of these three codons without affecting the amino acid composition of the encoded protein or the characteristics of the protein. Based upon the degeneracy of the genetic code, variant DNA molecules may be derived from the cDNA and gene sequences disclosed herein using a standard DNA mutagenesis techniques as described above, or by synthesis of DNA sequences. Thus, this invention also encompasses nucleic acid sequences that encode the polypeptides but that vary from the disclosed nucleic acid sequences by virtue of the degeneracy of the genetic code.

Transformed—A “transformed” cell is a cell into which a nucleic acid molecule has been introduced by molecular biology techniques. As used herein, the term “transformation” encompasses all techniques by which a nucleic acid molecule might be introduced into such a cell, including, but not restricted to, transfection with a viral vector, conjugation, transformation with a plasmid vector, and introduction of naked DNA by electroporation, lipofection, particle gun acceleration.

Nucleic Acid Constructs—Polypeptides of the invention can be produced by ligating a nucleic acid molecule encoding the polypeptide into a nucleic acid construct such as an expression vector, and transforming a bacterial or eukaryotic production cell with the expression vector. In general, nucleic acid constructs include expression control elements operably linked to a nucleic acid sequence encoding a polypeptide of the invention (e.g., lycopene e cyclase transferase A, B, or C). Expression control elements do not typically encode a gene product, but instead affect the expression of the nucleic acid sequence. As used herein, “operably linked” refers to connection of the expression control elements to the nucleic acid sequence in such a way as to permit expression of the nucleic acid sequence. Expression control elements can include, for example, promoter sequences, enhancer sequences, response elements, polyadenylation sites, or inducible elements.

In bacterial systems, a strain of E. coli such as DH10B or BL-21 can be used. Suitable E. coli vectors include, but are not limited to, pUC18, pUC19, the pGEX series of vectors that produce fusion proteins with glutathione S-transferase (GST), and pBluescript series of vectors. Transformed E. coli are typically grown exponentially then stimulated with isopropylthiogalactopyranoside (IPTG) prior to harvesting. In general, fusion proteins produced from the pGEX series of vectors are soluble and can be purified easily from lysed cells by adsorption to glutathione-agarose beads followed by elution in the presence of free glutathione. The pGEX vectors are designed to include thrombin or factor Xa protease cleavage sites such that the cloned target gene product can be released from the GST moiety.

In eukaryotic host cells, a number of viral-based expression systems can be utilized to express polypeptides of the invention. A nucleic acid encoding a polypeptide of the invention can be cloned into, for example, a baculoviral vector such as pBlueBac (Invitrogen, San Diego, Calif.) and then used to co-transfect insect cells such as Spodoptera frugiperda (Sf9) cells with wild-type DNA from Autographa californica multiply enveloped nuclear polyhedrosis virus (AcMNPV). Recombinant viruses producing polypeptides of the invention can be identified by standard methodology. Alternatively, a nucleic acid encoding a polypeptide of the invention can be introduced into a SV40, retroviral, or vaccinia based viral vector and used to infect suitable host cells.

A polypeptide within the scope of the invention can be “engineered” to contain an amino acid sequence that allows the polypeptide to be captured onto an affinity matrix. For example, a tag such as c-myc, hemagglutinin, polyhistidine, or Flag™ tag (Kodak) can be used to aid polypeptide purification. Such tags can be inserted anywhere within the polypeptide including at either the carboxyl or amino termini. Other fusions that could be useful include enzymes that aid in the detection of the polypeptide, such as alkaline phosphatase.

Agrobacterium—mediated transformation, electroporation and particle gun transformation can be used to transform plant cells. Illustrative examples of transformation techniques are described in U.S. Pat. No. 5,204,253 (particle gun) and U.S. Pat. No. 5,188,958 (Agrobacterium). Transformation methods utilizing the Ti and Ri plasmids of Agrobacterium spp. typically use binary type vectors. Walkerpeach, C. et al., in Plant Molecular Biology Manual, S. Gelvin and R. Schilperoort, eds., Kluwer Dordrecht, C1:1-19 (1994). If cell or tissue cultures are used as the recipient tissue for transformation, plants can be regenerated from transformed cultures by techniques known to those skilled in the art.

Production Cell—a cell that can be cultured such that it produces the carotenoids described herein and/or the polypeptides and nucleic acid sequences described herein. This includes, without limitation, prokaryotic cells such as R. sphaeroides cells and eukaryotic cells such as plant, yeast, and other fungal cells. It is noted that cells containing an isolated nucleic acid of the invention are not required to express the isolated nucleic acid. In addition, the isolated nucleic acid can be integrated into the genome of the cell or maintained in an episomal state. In other words, cells can be stably or transiently transfected with an isolated nucleic acid of the invention.

Any method can be used to introduce an isolated nucleic acid into a cell. In fact, many methods for introducing nucleic acid into a cell, whether in vivo or in vitro, are well known to those skilled in the art. For example, calcium phosphate precipitation, conjugation, electroporation, heat shock, lipofection, microinjection, and viral-mediated nucleic acid transfer are common methods that can be used to introduce nucleic acid molecules into a cell. In addition, naked DNA can be delivered directly to cells in vivo as describe elsewhere (U.S. Pat. Nos. 5,580,859 and 5,589,466). Furthermore, nucleic acid can be introduced into cells by generating transgenic animals.

Any method can be used to identify cells that contain an isolated nucleic acid within the scope of the invention. For example, PCR and nucleic acid hybridization techniques such as Northern and Southern analysis can be used. In some cases, immnunohistochemistry and biochemical techniques can be used to determine if a cell contains a particular nucleic acid by detecting the expression of a polypeptide encoded by that particular nucleic acid. For example, the polypeptide of interest can be detected with an antibody having specific binding affinity for that polypeptide, which indicates that cell not only contains the introduced nucleic acid but also expresses the encoded polypeptide. Enzymatic activities of the polypeptide of interest also can be detected or an end product (e.g., a particular carotenoid) can be detected as an indication that the cell contains the introduced nucleic acid and expresses the encoded polypeptide from that introduced nucleic acid.

The cells described herein can contain a single copy, or multiple copies (e.g., about 5, 10, 20, 35, 50, 75, 100 or 150 copies), of a particular exogenous nucleic acid. For example, a bacterial cell (e.g., Rhodobacter) can contain about 50 copies of an exogenous nucleic acid of the invention. In addition, the cells described herein can contain more than one particular exogenous nucleic acid. For example, a bacterial cell can contain about 50 copies of exogenous nucleic acid X as well as about 75 copies of exogenous nucleic acid Y. In these cases, each different nucleic acid can encode a different polypeptide having its own unique enzymatic activity. For example, a bacterial cell can contain two different exogenous nucleic acids such that a high level of a carotenoid is produced. In addition, a single exogenous nucleic acid can encode one or more polypeptides. For example, a single nucleic acid can contain sequences that encode three or more different polypeptides.

Microorganisms that are suitable for producing carotenoids may or may not naturally produce carotenoids, and include prokaryotic and eukaryotic microorganisms, such as bacteria, yeast, and fungi. In particular, yeast such as Phaffia rhodozyma (Xanthophyllomyces dendrorhous), Candida utilis, and Saccharomyces cerevisiae, fungi such as Neurospora crassa, Phycomyces blakesleeanus, Blakeslea trispora, and Aspergillus sp, Archaea bacteria such as Halobacterium salinarium, and Eubacteria including Pantoea species (formerly called Erwinia) such as Pantoea stewartii (e.g., ATCC Accession #8200), flavobacteria species such as Xanthobacter autotrophicus and Flavobacterium multivorum, Zymonomonas mobilis, Rhodobacter species such as R. sphaeroides and R. capsulatus, E. coli, and E. vulneris can be used. Other examples of bacteria that may be used include bacteria in the genus Sphingomonas and Gram negative bacteria in the α-subdivision, including, for example, Paracoccus, Azotobacter, Agrobacterium, and Erythrobacter. Eubacteria, and especially R. sphaeroides and R. capsulatus, are particularly useful. R. sphaeroides and R. capsulatus naturally produce certain carotenoids and grows on defined media. Such Rhodobacter species also are non-pyrogenic, minimizing health concerns about use in nutritional supplements. Streptomyces aeriouvifer, Bacillus subtilis, and Staphylococcus aureus also are suitable production cells. In some embodiments, it can be useful to produce carotenoids in plants and algae such as Haematococcus pluvialis, Dunaliella salina, Chlorella protothecoides, Zea mays, Brassica napus, Arabidopsis thaliana, Tagetes erecta, Lycopersicum esculentum, and Neospongiococcum excentrum.

It is noted that bacteria can be membranous or non-membranous bacteria. The term “membranous bacteria” as used herein refers to any naturally-occurring, genetically modified, or environmentally modified bacteria having an intracytoplasmic membrane. An intracytoplasmic membrane can be organized in a variety of ways including, without limitation, vesicles, tubules, thylakoid-like membrane sacs, and highly organized membrane stacks. Any method can be used to analyze bacteria for the presence of intracytoplasmic membranes including, without limitation, electron microscopy, light microscopy, and density gradients. See, e.g., Chory et al, (1984) J. Bacteriol., 159:540-554; Niederman and Gibson, Isolation and Physiochemical Properties of Membranes from Purple Photosynthetic Bacteria. In: The Photosynthetic Bacteria, Ed. By Roderick K. Clayton and William R. Sistrom, Plenum Press, pp. 79-118 (1978); and Lueking et al., (1978) J. Biol. Chem. 253: 451-457.

Examples of membranous bacteria that can be used include, without limitation, Purple Non-Sulfur Bacteria, including bacteria of the Rhodospirillaceae family such as those in the genus Rhodobacter (e.g., R. sphaeroides and R. capsulatus), the genus Rhodospirillum, the genus Rhodopseudomonas, the genus Rhodomicrobium, and the genus Rhodopila. The term “non-membranous bacteria” refers to any bacteria lacking intracytoplasmic membrane. Membranous bacteria can be highly membranous bacteria. The term “highly membranous bacteria” as used herein refers to any bacterium having more intracytoplasmic membrane than R. sphaeroides (ATCC 17023) cells have after the R. sphaeroides (ATCC 17023) cells have been (1) cultured chemoheterotrophically under aerobic condition for four days, (2) cultured chemoheterotrophically under anaerobic for four hours, and (3) harvested. Aerobic culture conditions include culturing the cells in the dark at 30° C. in the presence of 25% oxygen. Anaerobic culture conditions include culturing the cells in the light at 30° C. in the presence of 2% oxygen. After the four hour anaerobic culturing step, the R. sphaeroides (ATCC 17023) cells are harvested by centrifugation and analyzed.

II. Brief Overview

The present mvention involves the identification, isolation, and cloning of genes involved in a non-mevalonate pathway for carotenoid biosynthesis. In particular, the isolated genes allow for the biosynthesis of a C40 carotenoid and the conversion of the C40 carotenoid to a C50 carotenoid. The isolated genes can be introduced into a production cell. The production cell can be used to produce the polypeptides for use in vitro (outside of the cell) or the production cell can be used to make C>40 carotenoids, such as C50 carotenoids and various derivatives.

The identification of one set of representative genes allows for the isolation of genes that have similar nucleic acid and/or amino acid sequences, which have a similar function. The isolated genes offer an advance in the art, because they allow for the conversion of a C40 carotenoid to a C>40 carotenoid, such as a C50 carotenoid.

The nucleic acid sequences provided herein encode three separate polypeptides. An important finding of the invention is that the activity of all three polypeptides can be used to convert a C40 carotenoid to the C50 carotenoid. The nucleic acid molecules were first isolated from A. mediolanus. Similar genes with substantial homology were then isolated from M. lueus. The genes from M. lueus were also shown to be active. It is believed that other similar genes with substantial homology could be isolated from other bacteria using similar techniques, and that such genes fall within the present invention.

The present invention is particularly important because it provides a key step to the ability to convert carotenoids from the C40 level to the C50 level by genetic manipulation.

The invention uses standard laboratory practices, such as for the cloning, manipulation, and sequencing of nucleic acids, purification and analysis of proteins and other molecular biological and biochemical techniques, unless otherwise specified. Such standard techniques are explained in detail in standard laboratory manuals such as Sambrook et al., Molecular Cloning: A Laboratory Manual, 2nd edition., vol. 1-3, Cold Spring Harbor, New York, 1989; and Ausubel et al., Current Protocols in Molecular Biology, Greene Publ. Assoc. & Wiley-Intersciences, 1989.

III. Experimental Materials, Methods, Results, and Examples—Agronsyces mediolanus

Brief Outline of the Subject Matter Described in Section III

1. The selection of A. mediolanus as the bacterium for which genomic DNA would be extracted.

2. The construction of a genomic DNA library, the isolation of genomic colonies, and the selection of experimental working colonies. A particularly important experimental working colony was called Y1.

3. The isolation of a plasmid DNA from the Y1 colony, and the identification of a carotenogenic operon contained therein.

4. The sequencing and sequence analysis of the carotenogenic operon.

5. The identification of seven (7) genes (idi, crtE, crtB, crtI, lctA (ORF X1), lctB (ORF X2), and lctC (ORF Y) from the operon, wherein one or more of the seven (7) isolated genes allow for the biosynthesis of the C50 carotenoid and the conversion of a C40 carotenoid to a C>40 carotenoid, such as a C50 carotenoid. The identification included, among other aspects, the determination of the respective nucleic acid sequences and encoded amino acid sequences.

6. The creation of constructs of certain combinations of the seven genes. The constructs were amplified with primers and PCR. Deductive analysis was performed on the amplified constructs to determine the capabilities of individual constructs. The pathway of the associated biosynthetic reactions was determined. The portion of the pathway associated with individual genes was also determined.

7. The recognition that four of the previously unidentified genes (4) (idi, crtE, crtB, crtI) of the seven (7) isolated genes allow for the production of a C40 carotenoid, in a manner having certain similarities to techniques already known it the art.

8. The realization that three (3) (lctA, lctB, lctC) of the seven (7) isolated genes represented a significant advance to the art, because the genes allow for the conversion of a C40 carotenoid to a C>40 carotenoid, such as a C50 carotenoid.

9. The realization that the activities that are provided by the three (3) genes (lctA, lctB, lctC) can be used to convert a C40 carotenoid to a C50 carotenoid in a single step.

10. The cloning of certain constructs of the seven (7) isolated genes into host bacteria, which resulted in successful carotenogenic reactions.

Details elaborating the brief outline are described in the remainder of section III.

A. Selection of Agromyces mediolanus; Agromyces mediolanus genomic DNA Preparation

Flavobacterium dehydrogenans was chosen as the bacterial source for the identification of genes since the bacterium had been reported to produce both C40 and C50 carotenoids (Weeks OB et al. Nature 224:879-82, 1969). Since F. dehydrogenans was an unidentified bacterium in the ATCC (American Type Culture Collection), the strain was submitted for identification. Microbial identification revealed the organism to be Agromyces mediolanus. Although there were reports in the literature describing the production of the C50 carotenoid decaprenoxanthin in (F. dehydrogenans) A. mediolanus (Schwieter U, and Liaaen-Jensen S. Acta Chem Scand 23:1057, 1969, and Liaaen-Jensen S, et al. Acta Chem Scand 22:1171-86, 1968), no reports were found on the genes responsible for C50 carotenoid biosynthesis.

A. mediolanus was grown in 200 mL of nutrient broth for 36 hours at 30° C. and 250 rpm. Cultured cells were centrifuged to form a cell pellet, and washed by resuspending the pellet in a 10 mM Tris:1 mM EDTA (ethylene diaminetetraacetate) solution, and centrifuged again. The cell pellets were resuspended in 5 mL of GTE buffer (50 mM glucose, 25 mM Tris HCl, pH 8.0, 10 mM EDTA, pH 8.0) per 100 mL of culture. The bacterial cell walls were lysed by adding lysozyme and Proteinase K, each to a 1.0 mg/mL final concentration, and mutanolysin to a 5.5 μg/mL final concentration. After a 1.5 hours incubation at 37° C., SDS (sodium dodecyl sulfate) was added to a final concentration of 1% and the concentration of Proteinase K was brought to 2 mg/mL. After incubation at 50° C. for one hour, the solution containing the lysed cells was diluted 1:1 with fresh GTE buffer and NaCl was added to a 0.15 M concentration in the diluted solution. The mixture was extracted with an equal volume of phenol:chloroform:isoamyl alcohol (25:24:1) and centrifuged at 12,000×g for 10 minutes. The supernatant was removed and placed in a clean tube, extracted with an equal volume of chloroform, and centrifuged at 3,000×g for 10 minutes. The supernatant was treated with RNase and precipitated with 2.5 volumes of ethanol. After mixing the solution, the precipitated DNA was removed by spooling it on a glass rod. The spooled DNA was washed with 70% ethanol, air dried, and resuspended in 10 mM Tris, pH 8.5.

B. A. mediolanus genomic DNA Library Construction for Isolation of the Carotenoid Operon

A. mediolanus genomic DNA (80 μg) was digested at 37° C. for 10 minutes with 2.8 units of Sau3A I restriction enzyme (Promega, Madison, Wis.). The digested DNA was separated by gel electrophoresis using a 0.8% Tris-acetate-EDTA (TAE) agarose gel. DNA fragments ranging from 7-10 Kb in size were excised and purified using a Qiagen Gel Purification kit (Qiagen Inc., Valencia, Calif.). Vector to be used in the ligation (pUC19) was prepared by digesting with BamH I restriction enzyme (New England Biolabs, Inc., Beverly, Mass.), gel purifing, and dephosphorylating using shrimp alkaline phosphatase (Roche Molecular Biochemicals, Indianapolis, Ind.). BamHI DNA fragments (126 ng) were ligated into 50 ng of prepared pUC19 DNA at 14° C. for 16 hours using T4 DNA ligase (oche Molecular Biochemicals). The ligation reaction was precipitated by adding 1/10 volume 7.5 MNH₄OAc and 2.5 volumes ethanol, incubating at −20° C. for 3 hours, centrifuging to obtain a DNA pellet, washing the pellet with 70% ethanol, drying the pellet, and resuspending the pellet in 20 μL of 10 nM Tris buffer, pH 8.5. One microliter of ligation reaction was used to electroporate 40 μL of ElectroMAX™ DH10B™ competent cells (Life Technologies, Inc., Rockville, Md.). Electroporated cells were recovered in SOC media and plated on LB plates containing 100 μg/mL of ampicillin (LBA). The plating volume necessary to produce approximately 300 cells/plate was determined by plating various volumes of transformed cells. Using this information, 125 plates containing approximately 300 colonies each were plated from transformations using remains of the ligation reaction. Plates were incubated at 37° C. for one day and then at room temperature for one day. On the second day, one yellow colony (Y1) was identified and streaked to a new LBA plate. Plasmid DNA of this colony was isolated using a Qiaprep Spin Miniprep Kit (Qiagen, Inc.). EcoR I restriction digests (New England Biolabs, Inc.) of the plasmid DNA showed the plasmid to contain an insert approximately 9-Kb in size.

C. Subcloning and Sequencing of the A. mediolanus Carotenogenic Operon

Several restriction enzymes, including BamHI and Pst I, were used to digest 2 μg aliquots of plasmid DNA from the Y1 colony. A digest from BamHI produced two fragments approximately 9 Kb and 3 Kb in size and a digest from Pst I produced four fragments approximately 4.5, 3.0, 1.5, and 1.0 Kb in size. These fragments were gel purified, ligated into pUC19, and transformed into ElectroMAX™ DH10B™ competent cells as described above. The electroporated cells were plated on LB agar plates with 100 μg/mL of ampicillin and 50 μg/mL of 5-Bromo-4-Chloro-3-Indolyl-β-D-Galactopyranoside (gal, media=LBAX). Single, white colonies corresponding to each purified fragment were isolated. Plasmid DNA was isolated and used to obtain the DNA sequence of each insert, using either M13F and M13R vector primers or sequencing primers designed from internal DNA sequence. Individual sequences were aligned using the software Clone Manager and Align Plus (Scientific and Educational Software, Durham, N.C.).

D. Sequence Analysis of the A. mediolanus Carotenogenic Operon

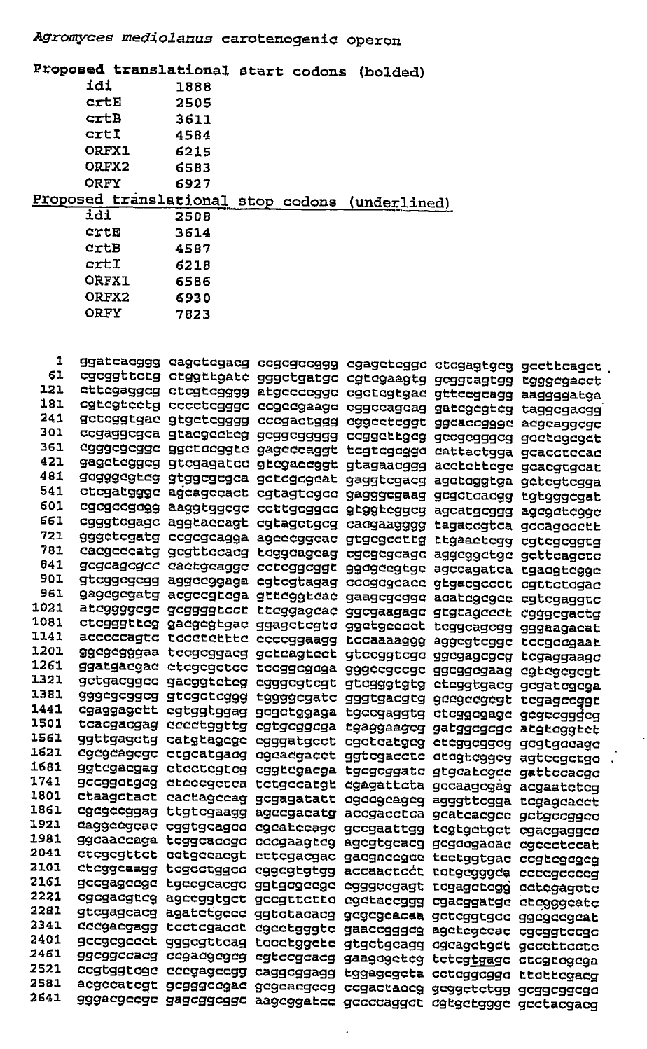

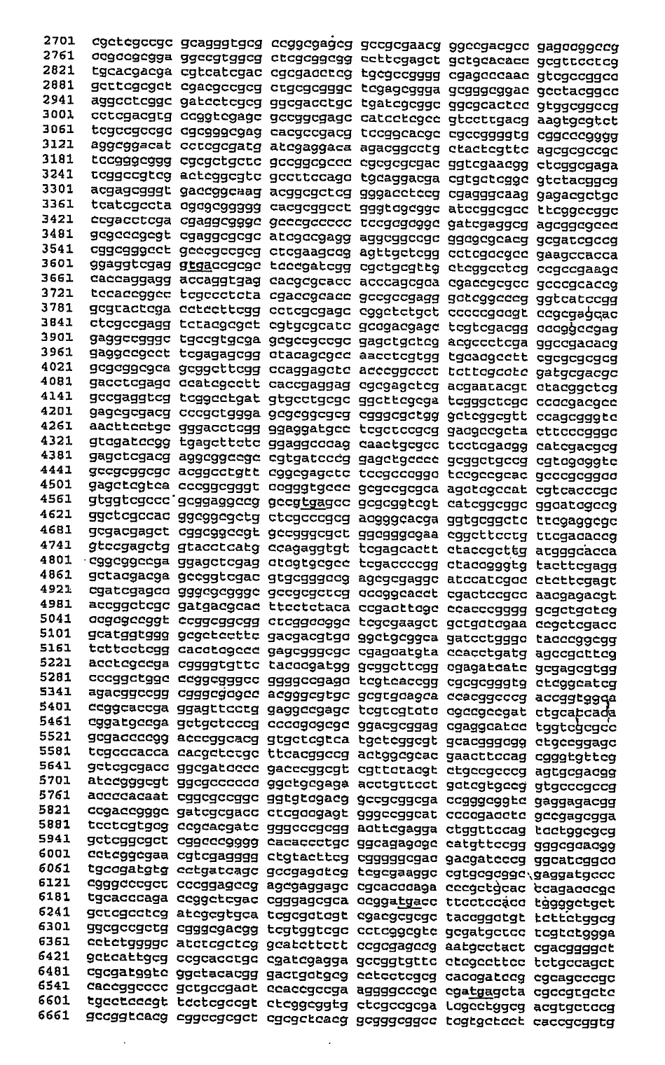

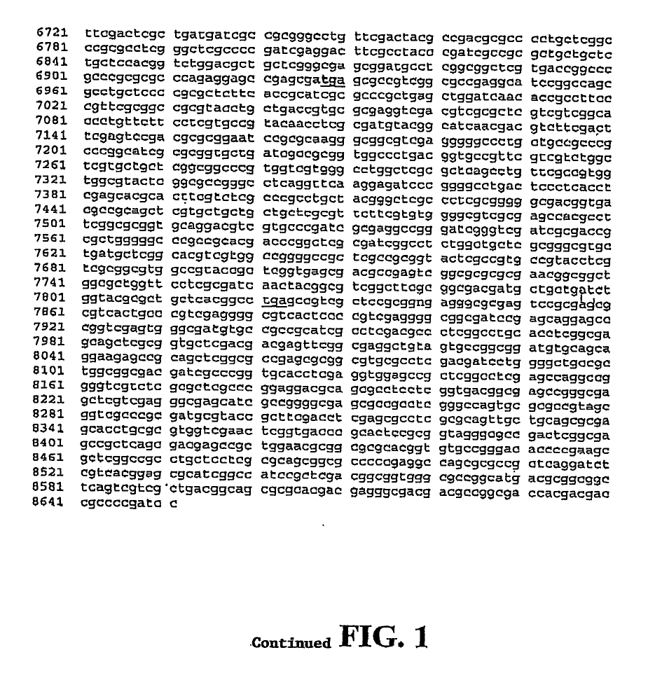

The BLAST DNA sequence comparison program (National Center for Biotechnology Information) was used to identify genes residing on the insert of the Y1 clone. The sequence of nucleotides residing on the insert of the Y1 clone was chosen as a working operon (the Y1 operon), and the location of the genes residing on the Y1 operon is shown in FIG. 1. The BLAST analysis identified the following genes, in order of location in the operon:
idi, isopentenyl pyrophosphate isomerase,
crtE, geranylgeranyl pyrophosphate synthase (CCPS synthese),
crtB, phytoene synthase, and
crtI, phytoene dehydrogenase (phytoene desaturase).

In addition, three open reading frames (ORFs) downstream of crtI were identified to which no definitive fluction could be assigned using sequence similarity. The three ORFs were given the following names:
ORFX1—the first ORF downstream of crtI—was 372 nucleotides in length
ORFX2—the second ORF downstream of crtI—was 348 nucleotides in length
ORFY—the third ORF downstream of crtI—was 897 nucleotide in length

ORFX1 showed homology (33% sequence identity) to the lycopene cyclase domain of the Rhizomucor carRP gene. The carRP gene encodes a polypeptide having both phytoene synthase and lycopene cyclase activities. Therefore, it is likely that the polypeptide encoded by the ORFX1 gene contributes cyclase activity during the conversion of lycopene to decaprenoxanthin.

No genes with significant homology were detected for ORFX2 in the Genbank database. The ORFY protein sequence had low homology with a DHNA-octaprenyltransferase from Bacillus subtilis in the Swisspro database. This enzyme catalyzes the attachment of a 40-carbon side chain to 1,4-dihydroxy-2-naphthoic acid (DHNA). BLAST searches of the ORFY DNA sequence to the NCBI non-redundant DNA database showed certain homology to ORFs identified in Deinococcus radiodurans, Halobacterium sp. NRC-1 (National Research Council of Canada, a cell repository), and Methanobacterium thermoautotrophicum. The Deinococcus radiodurans ORF in turn shows low homology to a Schizosaccharomyces pombe para-hydroxybenzoate polyprenyltransferase. The Halobacterium ORF shows significant homology to a Rhodobacter capsulatus bacteriochlorophyll synthase gene, which catalyzes the esterification of bacteriochlorophyll by geranylgeranyl-pyrophosphate, and low homology to a Saccharomyces cerevisiae para-hydroxybenzoate polyprenyltransferase.

E. A. mediolanus DNA Constructs for Carotenoid Production

1. The Constructs and Carotenoid Production

Initial data indicated that the inclusion of the idi gene in an expression vector was likely necessary to achieve detectable carotenoid expression levels. The initial experiments also indicated that the use of a medium copy number vector was preferable to use of a high copy number vector, possibly due to a detrinental effect on the bacterial cell of maintaining the latter. Therefore, the expression vector pProLarNde was used. This vector is a modification of the pPROLar.A vector (CLONTECH Laboratories, Inc., Palo Alto, Calif.) into which an Nde I restriction site was inserted downstream of the ribosomal binding site.

Primers were designed to amplify three regions of the Y1 operon: (a) the region from idi through crtI—the idi-crtI construct (4.6 KB), (b) the region from idi through ORFX2—the idi-ORFX2 construct (5.3 KB), and (c) the region from idi through ORFY—the idi-ORFY construct (6.7 Kb). These primers were designed to introduce an Nde I restriction site at the beginning of the amplified fragment and a Hind In restriction site at the end of the amplified fragment. The sequences of the primers were as follows, with the restriction sites underlined:

| | |
|---|---|
| Primer name | Primer sequence |

(SEQ ID NO: 27)

| | |
|---|---|
| AIDINDEF | 5′-TTCATATGTCACTAGCCAGGCGAGATATCC-3′ |

(SEQ ID NO: 28)

| | |
|---|---|
| APDHIIIR | 5′-GAAAGCTTAAGAAGATGCCGAGCGAGATG-3′ |

(SEQ ID NO: 29)

| | |
|---|---|
| AXHIIIR | 5′-AGAAGCTTTGTACGGCACGAGGAAGAACAG-3′ |

(SEQ ID NO: 30)

| | |
|---|---|
| AYHIIIR | 5′-GAAAGCTTCTCCGTGACGAGATCCTGAG-3′ |

Due to the high GC content of A. mediolanus, PCR was conducted using the Advantage®—GC Genomic Polymerase (CLONTECH) kit. The PCR reaction mix, according to manufacturer's specifications, used a 1.0 M final GC-Melt concentration and 1.0 ng of A. mediolanus genomic DNA per μL of reaction mix in a 100-200 μL reaction. The PCR reactions were performed in a Perkin Elmer Geneamp system 2400 under the following conditions: (a) an initial denaturation at 94° C. for 45 seconds; (b) 8 cycles of (1) 94° C. for 25 seconds, (2) 56° C. for 1 minute, and (3) 72° C. for 10 minutes; (c) 25 cycles of (1) 94° C. for 25 seconds, (2) 60° C. for 1 minute, and (3) 72° C. for 10 minutes; and (d) a final extension of 72° C. for 10 minutes. The PCR reactions were subjected to gel electrophoresis using a 0.8% TAE agarose gel. Fragments of the expected sizes were gel purified as previously described. Purified DNA was digested overnight with Hind III and Nde I to make the fragment ends compatible with digested pPROLarNde vector. The digested PCR product was purified using a Qiagen PCR Purification column and quantified on a spectrophotometer.

pPROLarNde vector (5 μg) was digested overnight with Hind m and Nde I and purified using gel electrophoresis on a 1% TAE agarose gel and a Qiagen Gel Purification Kit. The digested and purified vector was dephosphorylated using calf intestinal alkaline phosphatase (CIAP, Promega) according to manufacturer's specifications with the following exceptions: (a) 40 μL of eluent from the Qiagen purification was used directly as the starting DNA, (b) the CIAP was used at a 1/20 enzyme dilution rather than a 1/100 dilution, and (c) the dephosphorylated DNA was purified using a Qiagen PCR Purification Column rather than by ethanol precipitation.

The purified and digested PCR products were each ligated into 50 ng of prepared pPROLarNde DNA at 16° C. for 16 hours using T4 DNA ligase (Roche Molecular Biochemicals). One μL of each ligation reaction was used to electroporate 40 μL of ElectroMAXT DHIOBTM competent cells. Electroporated cells were recovered in SOC media for one hour and plated on LB plates containing 50 μg/mL of kanamycin, 1 mM isopropylthio-β-D-galactoside (IPTG), and 2% L-arabinose (LBKIA).

Two red colonies were isolated from E. coli transformed with the idi-crtI construct; two red colonies were isolated from E. coli transformed with the idi-ORFX2 construct; one yellow colony was isolated from E. coli transformed with the idi-ORFY construct. Each of these colonies had the desired insert size, as indicated by PCR and by restriction enzyme digest with Hind III and Nde I. DNA sequencing of the X1-X2-Y region was conducted on plasmid DNA from these colonies to check for PCR errors.

Carotenoids were extracted from 100 mL cultures grown for 3 days in LBKIA media at 30° C. and 200 rpm. Cells were pelleted by centrifugation at 12,000 g for 10 minutes, washed with sterile distilled water, and re-centrifuged. The pellet was dried and resuspended in 2 mL of acetone by vortexing in the presence of glass beads. The extraction of the carotenoids was performed at 55° C. for a total of 1.5 hours and at room temperature for one hour. Extractions were conducted in the dark to prevent light-induced degradation of carotenoids, and with vortexing every 15 minutes to enhance cell exposure to the solvent. The extraction mixture was then centrifuged at 27,00 g for 15 minutes to obtain a hard pellet of cell matter. The supernatant of the carotenoids was passed through a 0.2 micron filter and the absorption curve from 400600 nm was read on a Cary 100 spectrophotometer.

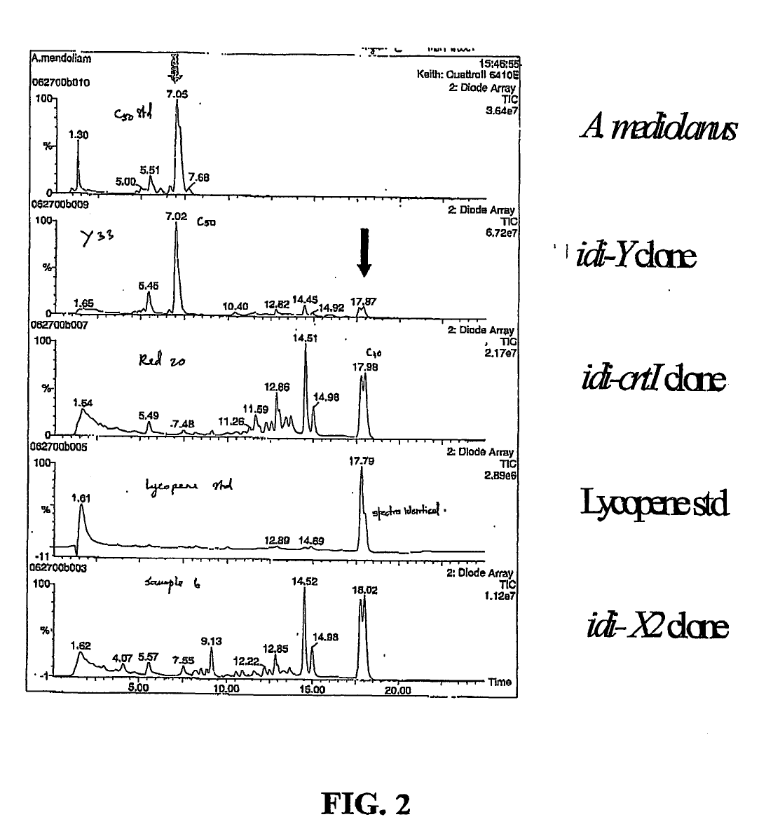

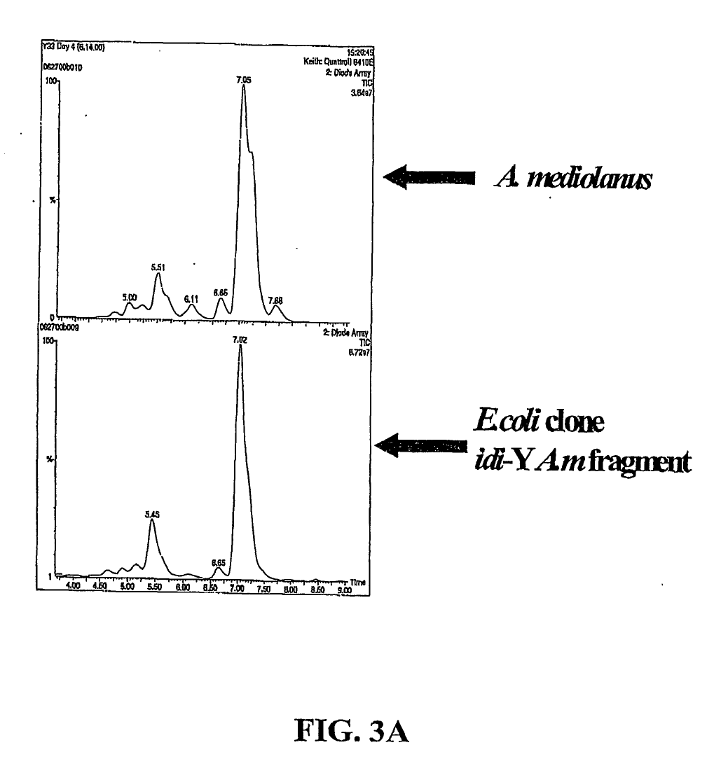

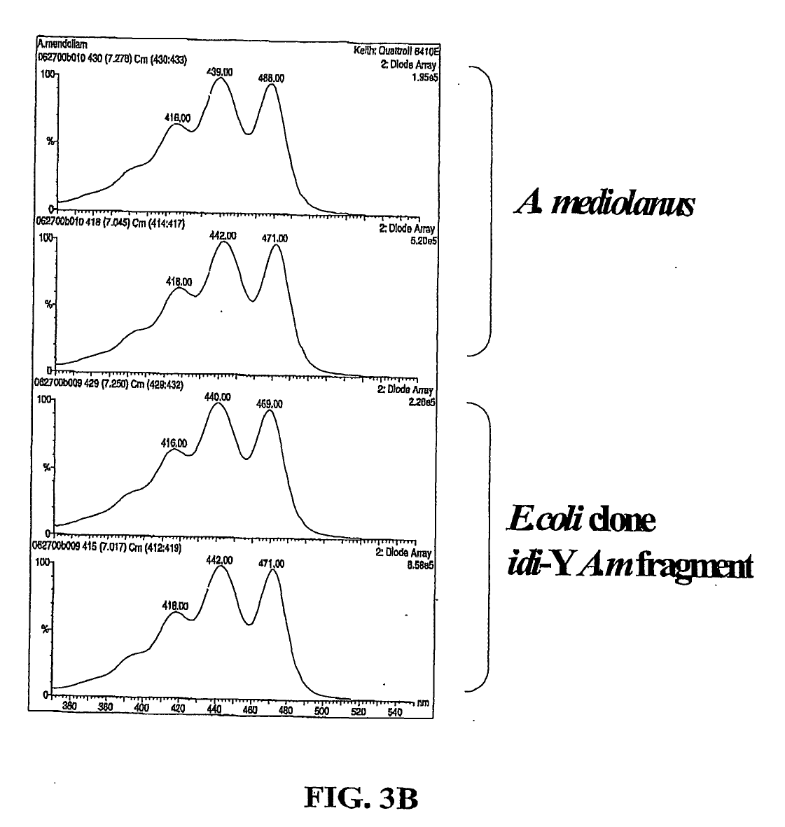

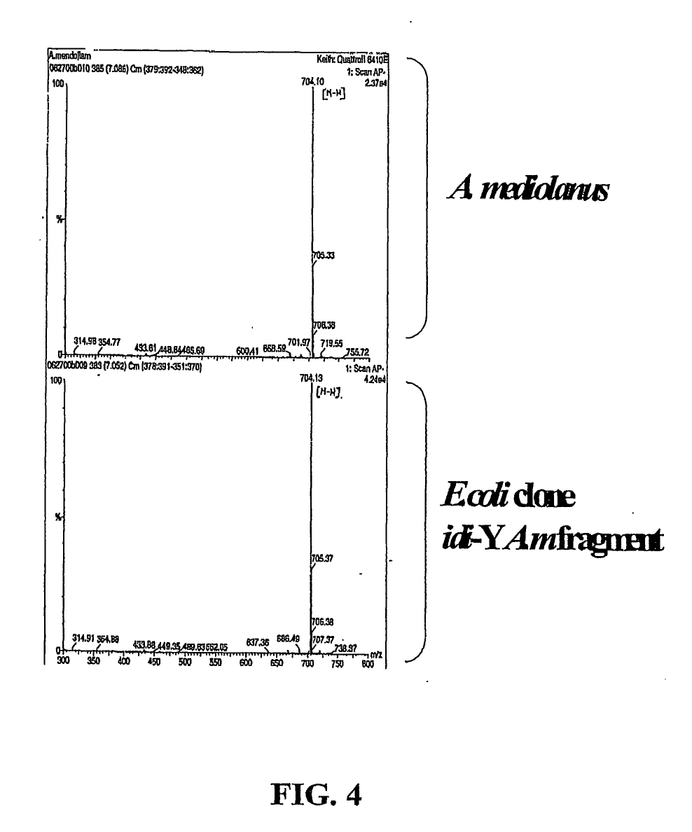

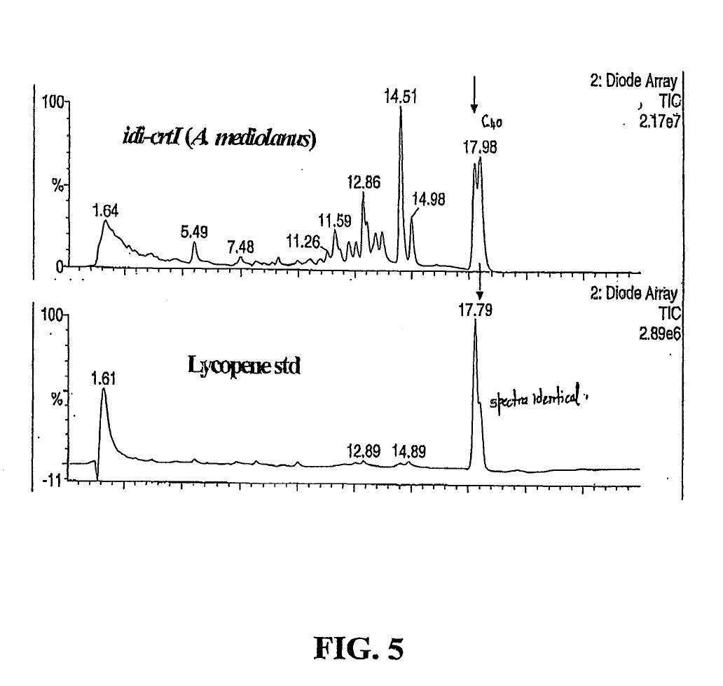

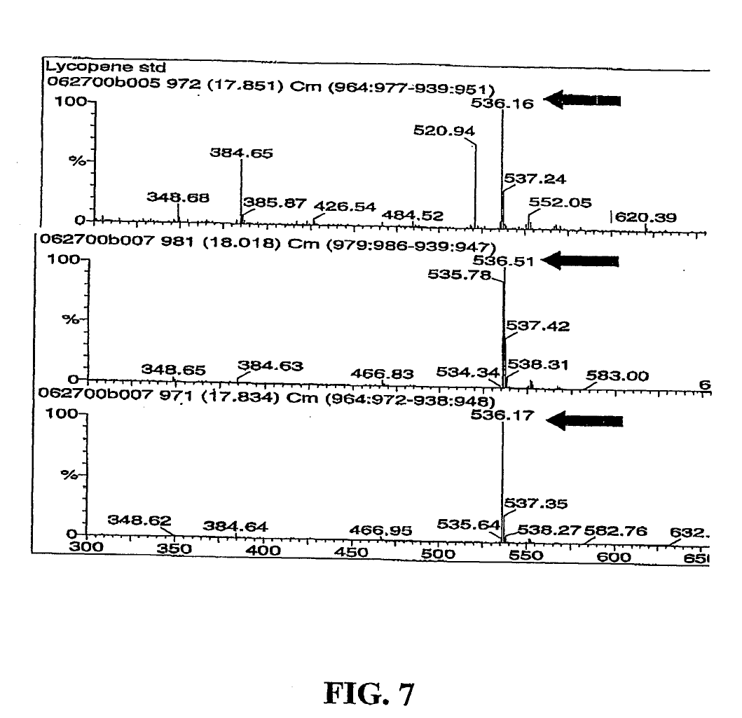

HPLC analysis of the carotenoid extracts from various clones is shown in FIG. 2 and FIG. 3. It is significant that the C50 carotenoid extracted from the E. coli clone with the idi-Y A. mediolanus fragment showed a mass that was identical to that observed in A. mediolanus wild type extract (FIG. 4). Absorption curves showed that the carotenoid material produced from E. coli containing the idi-crtI construct and the carotenoid material produced from E. coli containing the idi-ORFX2 construct have a spectrum identical to that of lycopene (a C40 carotenoid) (FIG. 5). HPLC analysis of the extracts and mass spectrometric analysis confirmed these observations (FIG. 7).

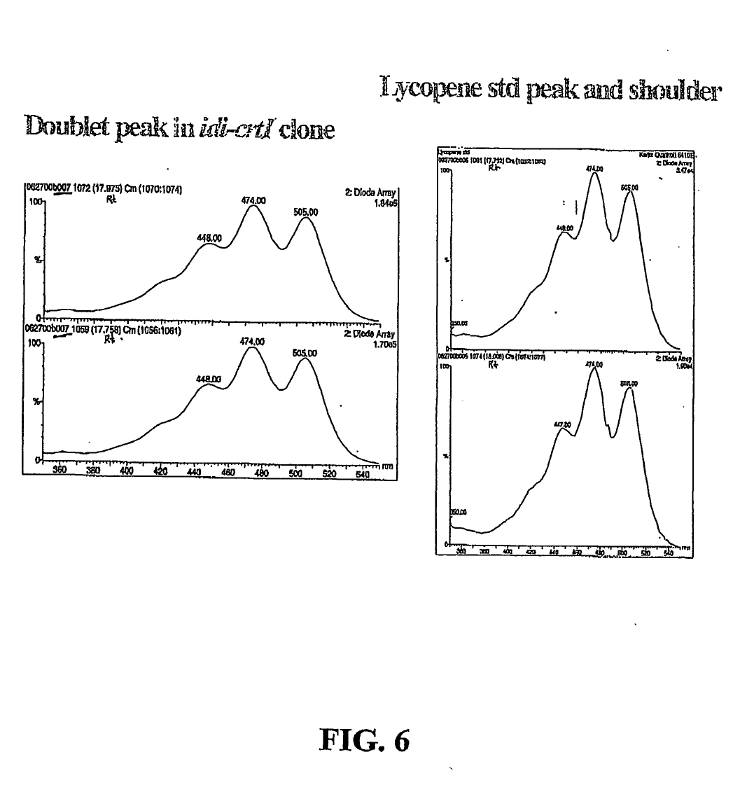

The carotenoid material produced from the idi-ORFY construct exhibited a spectrum that appeared to be a mixture of carotenoids, including both lycopene (FIG. 6) and the C50 carotenoid produced by the original Y1 clone (FIG. 3B).

2. The Relationship of ORFX1, ORFX2, and ORFY to the Production of the C50 Carotenoid

The production of the C50 carotenoid by the E. coli clone having the idi-ORFY construct and lack of production by the clone having the idi-ORFfX2 construct indicate that ORFY was necessary for production of the Y1 C50 carotenoid. To help determine whether the X1 and X2 ORFs were also necessary for production of the C50 carotenoid, the following strategies were employed:

The first strategy is detailed in Example 1, and it involved cloning ORFY into the idi-crtI/pPROLarNde construct to determine if the C50 carotenoid could be produced in the absence of the X1 and X2 ORFs. Primers for the amplification of ORFY were designed to introduce a Pac I restriction site at the beginning of the amplified fragment and an Xba I restriction site at the end of the amplified fragment, which would insert the ORFY fragment downstream of the idi-crtI genes. The sequences of the primers were as follows, with the restriction sites underlined:

| AYPACF | |
|---|---|
| 5′- | (SEQ ID NO: 31) |
| GTCTTAATTAACTGCTGCTCTGCTCCACGGTCT- | |
| 3′ | |
| AYXBAR | |
| 5′-TATCTAGACGCTCCGTGACGAGATCCTGAG- | (SEQ ID NO: 32) |
| 3′ | |

The PCR reaction mix contained 1× Pfu buffer, 0.2 mM each DNTP, 5% dimethyl sulfoxide (DMSO), 0.5 μM each primer, 10 units of Pfu DNA polymerase (Stratagene) and 200 ng of A. mediolanus genomic DNA in a 200 μL reaction. The PCR reactions were performed in a Perkin Elmer Geneamp system 2400 under the following conditions: an initial denaturation at 94° C. for 1 minute, 8 cycles of (1) 94° C. for 30 seconds, (2) 57° C. for 45 seconds, and (3) 72° C. for 3.5 minutes; 25 cycles of (1) 94° C. for 30 seconds, (2) 62° C. for 45 seconds, and (3) 72° C. for 3.5 minutes; and a final extension of 72° C. for 7 minutes. The PCR reactions were subjected to gel electrophoresis using a 1.0% TAE agarose gel. A fragment of the expected size was gel purified as previously described. Purified DNA was digested overnight with Pac I, purified using a Qiagen PCR purification column, digested for 3.5 hours with Nde I restriction enzyme, purified with a Qiagen PCR purification column, and eluted in 30 μL of 10 mM Tris.

The idi-crtI construct was similarly digested with Pac I and xba I, dephosphorylated with shrimp alkaline phosphatase (Roche, Basil, Switzerland), and gel purified. Eighty μg of the digested and purified idi-crtI construct was ligated with 120 ng of the ORFY product using T4 DNA ligase at 16° C. for 16 hours. A control ligation with no insert DNA was also performed. One microliter of each ligation reaction was used to transform E. coli ElectroMAX™ DH10B™ competent cells. The transformation reactions were recovered in 300 μL of SOC media for 1 hour and plated on both LB media with 50 μg/bL kanamycin (LBK) and LBKIA media. Several colonies that grew on the LBK plates were patched to LBKIA plates. Plasmid DNA was isolated from single colonies and shown to have the desired insert size through digestion withXba I restriction enzyme.

The second strategy used a two-vector system. ORFY was cloned into the Sph I/Xba I sites of pUC19 and used in double transformations with the idi-crtI/pPROLarNde vector. Plasmid DNA was isolated from single colonies and digested withXba I and anXba I/Sph I mix to check the insert size. Electrocompetent cells of E. coli strain DH5αPRO (CLONTECH) were transformed with both the idi-crtI/pPROLarNde vector and the ORFY/pUC19 vector in a 5:1 ratio due to a lower transformation rate of the first vector. Cells were recovered in SOC media for 1 hour and plated on LB media containing 100 μg/nL ampicillin and 50 μg/M1 kanamycin (LBAK) and LBKIA media with 100 μg/mL ampicillin (LBAKIA). Single colonies were patched to new LBAKIA plates. All resulting colonies were red in color. Plasmid DNA was isolated from double transformants and digested with Xba I to check the size of both plasmids. Carotenoids were extracted from the clones and identified as lycopene (a C40 carotenoid) on the basis of the visible spectral profile.

The experiments described in the first and second strategies indicate that the idi-crtI construct with the addition of ORF Y—but without ORFX1 and ORFX2—can produce C40 carotenoids but did not produce C50 carotenoids.

The third strategy is detailed in Example 3 and involves site-directed mutagenesis to introduce frameshift mutations individually in ORFX1, ORFX2, and ORFY to help determine if the X1 and X2 ORFs were needed for production of the Y1 C50 carotenoid. A plasmid containing the X1, X2, and Y ORFs in pUC19 was constructed as follows and used as template for mutagenic PCR. The QuikChange™ Site-Directed Mutagenesis Kit (Stratagene, La Jolla, Calif.) was then used to produce a vector containing a mutation in ORFX1, a vector with a mutation in ORFX2, and a vector containing a mutation in ORFY. Primers were designed to amplify the region of A. mediolanus genomic DNA containing the X1, X2, and Y ORFs. These primers were designed to introduce an Sph I restriction site at the beginning of the amplified fragment and anXba I restriction site at the end of the amplified fragment. The sequences of the primers were as follows, with the restriction sites underlined:

| AXSPHF | |
|---|---|
| 5′-TAGGCATGCAACGTCGAGGGGCTGTACTTC- | (SEQ ID NO: 33) |
| 3′ | |
| AYXBAR | |
| 5′-TATCTAGACGCTCCGTGACGAGATCCTGAG- | (SEQ ID NO: 32) |
| 3′ | |

As part of the third strategy, the non-mutated ORFX1, ORFX2, ORFY fragment was combined with an idi-crtI fragment. This was done using PCR conducted using the Advantage®—GC Genomic Polymerase (CLONTECH) Kit. The PCR reaction mix was according to manufacturer's specifications, using a 1.0 M final GC-Melt concentration and 1.0 ng of A. mediolanus genomic DNA per μl of reaction mix in a 100-200 μL reaction. The PCR reactions were performed in a Perkin Elmer Geneamp system 2400 under the following conditions: an initial denaturation at 94° C. for 1 minute, 8 cycles of (1) 94° C. for 30 seconds, (2) 56° C. for 45 seconds, and (3) 72° C. for 3.75 minutes; 25 cycles of (1) 94° C. for 30 seconds, (2) 60° C. for 45 seconds, and (3) 72° C. for 3.75 minutes; and a final extension of 72° C. for 7 minutes. The PCR reactions were subjected to gel electrophoresis using a 1.0% TAE agarose gel. Fragments of the expected size were gel purified as previously described. Purified DNA was digested overnight with Xba I and Sph I restriction enzymes to make the fragment ends compatible with digested vector and purified using a Qiagen PCR Purification column.

The pUC 19 vector was digested with Sph I and Xba I, gel purified, and dephosphorylated as described previously. The digested and purified vector (65 ng) was ligated with 360 ng of the X1X2Y insert using T4 DNA ligase at 16° C. for 16 hours. A control ligation with no insert DNA was also performed. One microliter of each ligation reaction was used to transform E. coli ElectroMAX™ DH10B™ competent cells. The transformation reaction was recovered in 300 μL of SOC media for 1 hour and plated on LBAX media. Single, white colonies were screened by PCR to determine if they contained the desired insert. Plasmid DNA was isolated from seven colonies positive for the insert. Equal amounts of DNA of each of the seven plasmids was pooled. 25 ng of the pooled X1X2Y/pUC19 plasmid DNA and 100 ng of idi-crtI plasmid DNA were transformed into electrocompetent cells of the E. coli strain DH5αPRO. Cells were recovered for 1 hour in SOC media and plated on LBAK and LBAKIA media. The resulting colonies were either yellow or red, with red colonies presumably resulting from errors in DNA replication during PCR of the X1X2Y fragment. Plasmid DNA was isolated for three yellow colonies and exhibited the desired inserts upon digestion with Xba I. Carotenoid extractions on these three cultures showed that they were producing the C50 carotenoid of the original Y1 clone. Thus, the non-mutated ORFX1, ORFX2, ORFY fragment combined with the idi-crtI fragment was capable of producing a C50 carotenoid when introduced into E. coli.

As another part of the third strategy, mutated ORFX1, ORFX2, and ORFY fragments were individually combined with an idi-crtI fragment.

The following primers were used in mutagenesis:

(SEQ ID NO: 34)

| | |
|---|---|
| X1A | 5′-GCTCGTCGACGCGCGCTAGCCGGCTGTTCTTCTGG-3′ |

(SEQ ID NO: 35)

| | |
|---|---|
| X1B | 5′-CCAGAAGAACAGCCGGCTAGCGCGCGTCGACGAGC-3′ |

The underlined base was inserted, causing a frameshift mutation and creating a unique Nhe I site in the plasmid.

In addition, a C nucleotide and a G nucleotide were deleted, respectively, from the spaces in the X2A primer and a C nucleotide and a G nucleotide were deleted, respectively, from the spaces in the X2B primer. The first mutation introduced a frameshift and a unique ANe I site, while the second mutation eliminated a potential translational start codon.

| | | |
|---|---|---|
| X2A | 5′-GGAACGGGAGGCAGAGCA GGC | (SEQ ID NO: 36) |
| | TAGCTCATCGGCGGGCCCTTCG-3′ | |
| X2B | 5′-GGGCCCGCCGATGAGCTA GCC | (SEQ ID NO: 37) |
| | TGCTCTGCCTCCCGTTCC-3′ | |

A G nucleotide was deleted from the space in the YA primer and a C was deleted from the space in the YB primer, in order to create a frameshift and a unique Nhe I site.

| | | |
|---|---|---|
| YA | 5′-GTGTTGATCCAGCT | (SEQ ID NO: 38) |
| | AGCGGGCGCGATGCGGTGAAG-3′ | |
| YB | 5′-TTCACCGCATCGCGCCCGCT | (SEQ ID NO: 39) |
| | AGCTGGATCAACACC -3′ | |

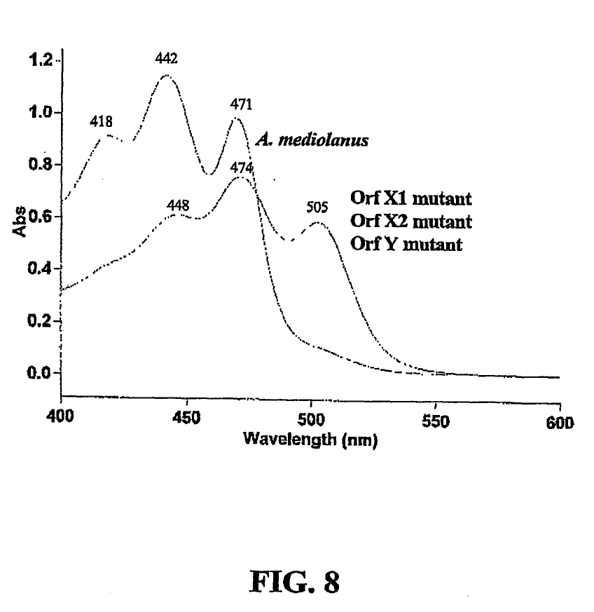

Mutagenic PCR was conducted using CLONTECH's Genome Advantage 5× Buffer, 1.0 M GCMelt, 1.1 mM MgOAc, 0.2 mM each dNTP, 15 ng of template DNA, and 2.5 units of Pfu Turbo DNA polymerase (Stratagene,) in a 50 μl reaction. Plasmid DNA ofthe X1X2/pUC19 construct, described above, was used as template. PCR was conducted according to the manufacturer's specification in the QuikChange™ Site-Directed Mutagenesis Kit, using a 14 minute extension time and 18 cycles of PCR Dpn I treatment and transformation were conducted as per manufacturer's specifications except that 2 μl of Dpn I-treated DNA was used in each transformation and cells were recovered in SOC media for 0.5 hour. Cells were plated on LBA plates and plasmid DNA was isolated from ten single colonies of each mutant type. Plasmid DNA of each colony was digested with Nhe I restriction enzyme to check for the introduction of a Nhe I site introduced through the mutagenic primer. All but one colony had a single Nhe I site, compared to the lack of a site in the X1X2Y/pUC19 template plasmid. The presence of the desired mutations and lack of unwanted mutations in other ORFs (i.e., an unwanted mutation in the Y ORF in the X1 mutation vector), were confirmed by sequencing. Plasmid DNA from two mutant colonies for the X1 mutation and one mutant colony for the X2 and Y mutations were used, along with the idi-crtI/pPROLarNde vector, in double transformations of electrocompetent cells of E. coli strain DH5αPRO. Control transformations using the unmutated X1X2Y/pUC19 vector and the idi-crtI/pPROLarNde vector were also conducted. All transformations used 25 ng of the pUC19-based vector and 100 ng of the pPROLarNde-based vector. Cells were recovered for one hour in SOC media and plated on LBAKIA media. Colonies from all of the transformations involving mutant plasmids were red, whereas the control double transformants were yellow. Visible spectral analysis revealed that all the mutant clones (red) produced the C40 carotenoid lycopene while the control double transformant and A. mediolanus (yellow) produced the C50 carotenoid decaprenoxanthin (FIG. 8).

Hence it was concluded that none of the fragments with mutations in ORFX1, ORFX2 or ORFY, combined with idi-crtI fragment were capable of producing a C50 carotenoid.

The results of the three strategies combined with the results from the tests of the previous three constructs (idi-crtI, idi-ORFX2, and idi-ORFY) indicate a significant finding—that the activities of all three ORFs can be used to convert a C40 carotenoid to a C50 carotenoid. If the genes of all three separate ORFs were not present, the conversion of the C40 carotenoid to a C>40 carotenoid was found to not occur.

3. The Naming of the ORF Genes which Allow for the Conversion of a C40 Carotenoid to a C50 Carotenoid

Because the ORFX1, ORFX2, and ORFY genes were all required for the conversion of the C40 lycopene (an acyclic carotenoid) to the C50 decaprenoxanthin (a carotenoid having two ε-ionone rings), the genes have been designated as lycopene ε-cyclase transferases, as described in the following table:
ORFX1 is designated lycopene β-cyclase transferase A, or ictA.
ORFX2 is designated lycopene ε-pyclase transferase B, or lctB.
ORFY is designated lycopene F—yclase transferase C, or lctC.

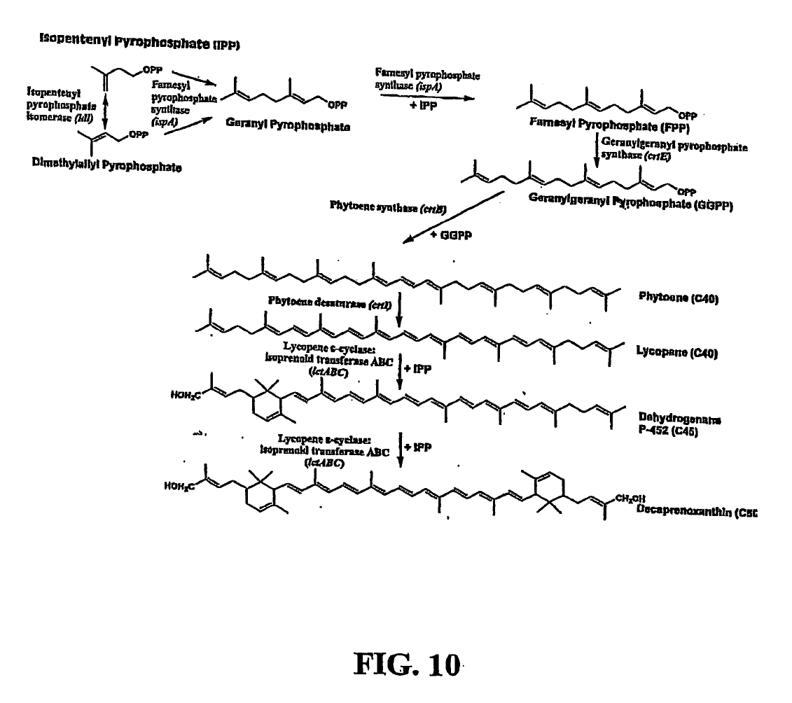

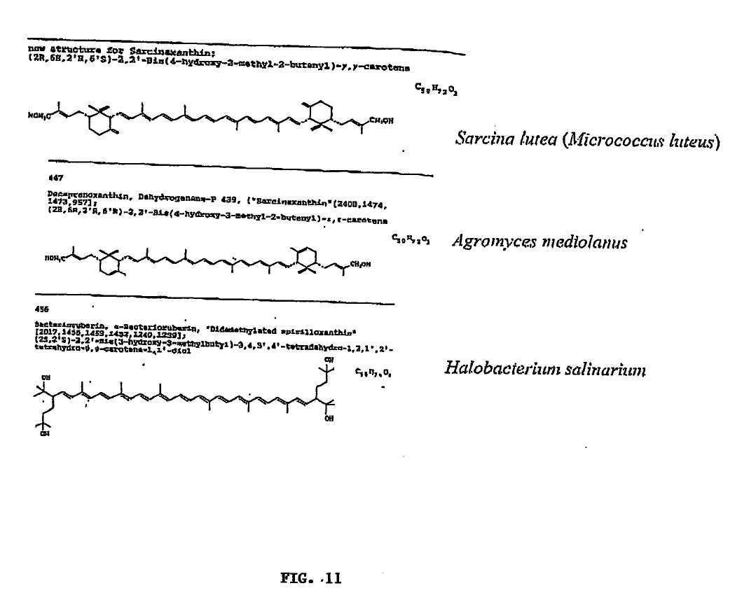

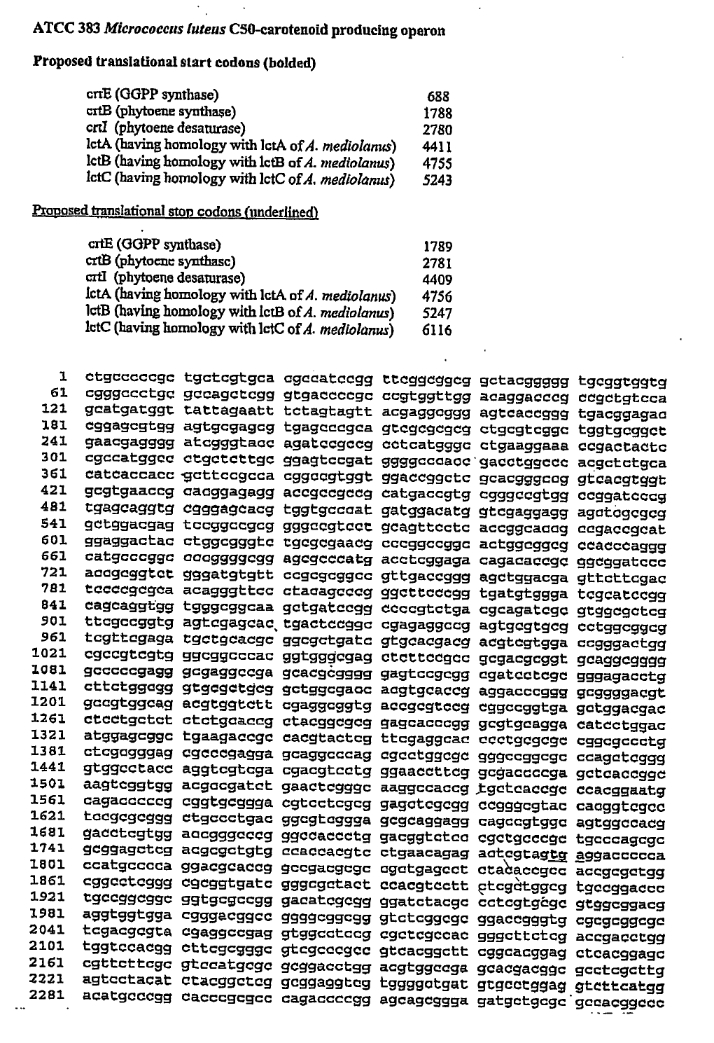

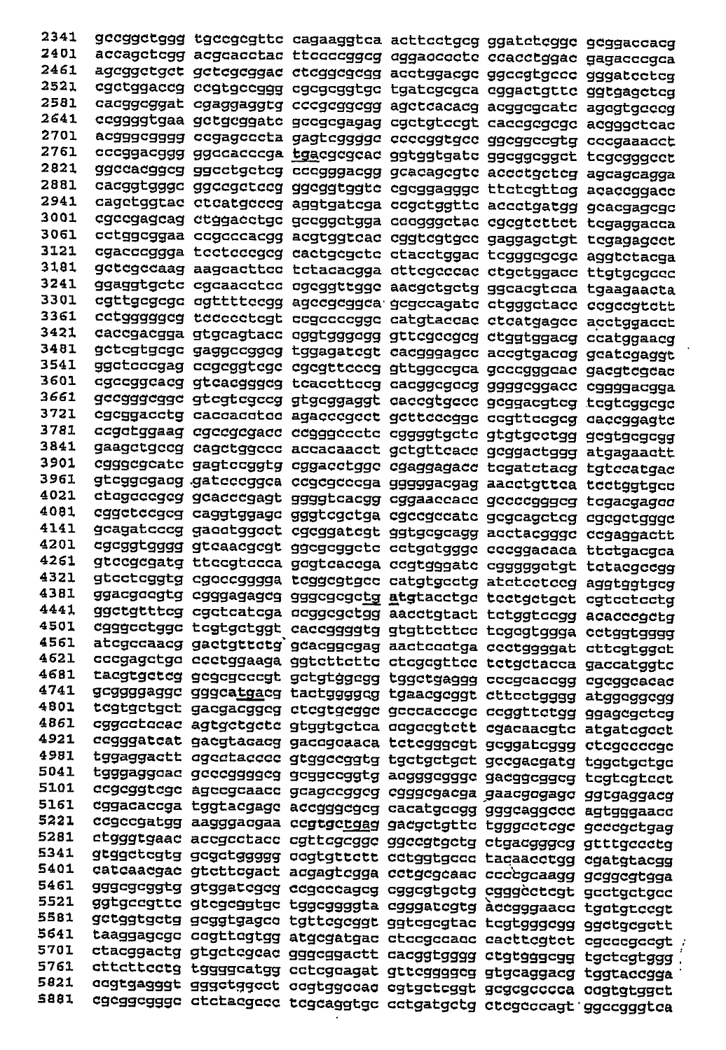

Based on the data described herein, a biosynthetic pathway for decaprenoxanthin in A. mediolanus is shown in FIG. 10. It is believed that the genes described herein could be present in other C50 producing bacteria such as Sarcina flava, Corynebacterium poinsettiae, Arthrobacter sp., such as A. glacialis, Sarcina luteus (Micrococcus luteus), Halobacterium cutirubram and salinarium, and Cellulomonas biazotea. It is believed that such genes could be isolated using techniques similar to those used for the present invention, and accordingly, such genes are considered part of the present invention.

IV. Experimental Materials, Methods, Results, and Examples—Micrococcus luteus

Brief Outline of the Subject Matter Described in Section IV

1. Selection of five CSO carotenoid producing bacteria as candidates for study; isolation of genomic DNA.

2. Synthesis of A. mediolanus lctC probe from previously described colony Y1.

3. Determination of homology between genes from each candidate bacterium and the lctC probe of A. mediolanus.

4. Selection of M. lueus ATCC 383 for study in view a substantial homology finding of one of its genes with the lctC probe.

5. Construction of a genomic DNA library for M. lueus ATCC 383.

6. Finding substantial homology between lctA, lctB, and lctC of M. lueus ATCC 383 and lctA, lctB, and lctC of A. mediolanus.

7. Identification of the carotenogenic operon for M. lueus ATCC 383.

8. Sequencing and sequence analysis for the carotenogenic operon.

9. Identification of six genes (crtE, crtB, crtI, lctA, lctB, and lctC) within the operon.

10. C50 production in M. lueus ATCC 383

11. BLAST analyses; Determining homology between genes.

Details elaborating the brief outline are described in the remainder of section IV.

A. Preparation of Genomic DNA for Candidate Bacteria; Choice of Micrococcus luteus (ATCC 383)

Five bacteria (species and strains) that produce C50 carotenoids were obtained from ATCC:\
Micrococcus luteus ATCC 147.
Micrococcus luteus ATCC 383.
Cellulomonas biazotea ATCC 486.
Halobacterium salinarium ATCC 33170.
Halobacterium salinarium NRC-1.
   In addition, the following control was employed
   Agromyces mediolanus ATCC 13930 (control).

Genomic DNA was isolated from each line plus the A. mediolanus control, using a Gentra Puregene DNA Isolation Kit (Gentra, Minneapolis, Minn.). Genomic DNA (1.0-1.5 μg) was used in digests with the restriction enzymes Pst I and Xho I, and separated on a 0.8% Tris-Acetate-EDTA (TAE) agarose gel. DIG-labeled molecular weight markers II and III (Roche Biomedical Products, Indianapolis, Ind.) were also included on the gel/membrane. DNA was transferred to a nylon membrane using a routine Southern transfer procedure.

DIG-labeled probes (894 bp) of the A. mediolanus lctC locus were synthesized using a PCR DIG Probe Synthesis Kit (Roche). Half-strength and full-strength DIG probes were amplified using plasmid DNA of the previously described Y1 clone as template and the ORFYF and ORFYR primers in 50 μL PCR reactions. The 5′ end of the ORFYF primer is located 14 bp upstream of the lctC translational start codon and the 5′ end of the ORFYR primer is located 15 bp upstream of the lctC translational stop codon.

| | | |
|---|---|---|
| ORFYF: | 5′-AGAGGAGCCGAGCGATGAG-3′ | (SEQ ID NO: 40) |
| ORFYR: | 5′-CGTACCAGATCAGCAGCATC-3′ | (SEQ ID NO: 41) |

The PCR reactions were separated on a 1% TAE-agarose gel and the probes were gel purified using a QIAquick Gel Purification Kit (Qiagen, Valencia, Calif.). After baking, membranes were prehybridized in EasyHyb Buffer (Roche) for at least 2 hours at 42° C. and hybridized overnight at 42° C. using 400 nL of the half-strength DIG labeling reaction per mL of hybridization solution. Washing of the membranes and detection of hybridization was achieved using a Wash and Block Buffer Set (Roche). Membranes were washed two times for 5-10 minutes each at room temperature in 2× SSC/0.1% SDS and two times for 15-20 minutes each at 55° C. in 0.l× SSC/0.1% SDS. After rinsing with washing buffer, the membranes were covered with blocking buffer and placed on a shaker for 1.5 hours at room temperature. The blocking buffer was replaced with fresh blocking buffer containing 150 mU of AP conjugate per mL of buffer and shaken at room temperature for an additional 30 minutes. Membranes were then washed twice for 15 minutes each at room temperature with washing buffer, followed by a five minute wash with detection buffer. The detection buffer was replaced with fresh detection buffer containing 20 μL of NBT/BCIP solution per mL of buffer. This was placed in the dark at room temperature with no shaking until color developed, after which the buffer was replaced with 10 mM Tris-1 mM EDTA solution.

Of the five strains tested, M. lueus ATCC 383 and M. lueus ATCC 147 showed fragments having the highest homology to the lctC probe. Restriction digests were done of genomic DNA of these two genotypes and A. mediolanus using the enzymes Xho I, ApaL I, and Sac I. DNA was separated on a 0.8% TAE-agarose gel, transferred to nylon membrane, and hybridized with the lctC probe as described above with the following exceptions. DIG-labeled Marker VII was included on gels/membranes. The DIG-labeled probe, which had been stored at −20° C., was heated at 65° C. for 15 minutes before reuse. After two washes in 2× SSC/0.1% SDS, membranes were washed twice at 64° C. in 0.5× SSC/0.1% SDS.

Whereas M. lueus ATCC 147 exhibited multiple bands of hybridization, M. luteus ATCC 383 showed a single dominant band for most of the digests. The Sac I digest for M. lueus exhibited a relatively strong band of approximately 4 Kb. Multiple Sac I digests were done for this genotype and separated on a 0.8% TAE-agarose gel. DNA fragments approximately 3.5-4.5 Kb in size were excised and gel purified using a QIAquick Gel Purification Kit.

In view of the above findings, M. lueus ATCC 383 was chosen for furer study.

B. Library Construction for M. lueus 383; Identification of the Carotenogenic Operon

The pUC18 vector (2.5 μg) was digested for 3 hours using Sac I restriction enzyme to generate fragment ends compatible with the digested genomic DNA from M. luteus ATCC 383. The Sac I-digested pUC 8 was dephosphorylated using shrimp alkaline phosphatase (SAP, Roche Diagnostics GmbH) and subsequently purified using gel electrophoresis on a 0.8% TAE-agarose gel and a QIAquick Gel Purification kit as per the manufacturer's instructions.

Purified insert DNA (60 ng) was ligated with 40-140 ng of prepared vector using T4 DNA ligase at 16° C. for 16 hours. A portion of the ligation reaction (1.2 μL) was electroporated into 40 μL of E. coli Electromax™ DH10B™ cells using standard electroporation protocols. Transformations were plated on LB media containing 40 μg/mL of X-gal and 100 μg/mL of carbenicillin (LBCX). Once an appropriate plating volume was determined, multiple transformations were conducted using remaining portions of the ligation reaction and were plated to achieve individual colonies.

Individual, white colonies were patched in a 6×7 grid to 14 plates of LB with 100 μg/mL of carbenicillin (LBC). Upon growth, colonies were replica plated to new LBC media. Colony lifts were made, according to standard procedures, using one of the sets of plates. Plasmid DNA of the A. mediolanus Y1 colony (5 ng) was spotted to some of the membranes as a hybridization control. After baking, each membrane was treated with 600 μL of 1.67 mg/mL Proteinase K (Qiagen) diluted in 2× SSC and heated at 37° C. for 1.25 hours. Membranes were then rinsed in 2× SSC on a shaker for one hour at room temperature. Prehybridization, hybridization with the lctC probe, membrane washing, and detection of hybridization were conducted as previously described.

Twelve colonies were identified that hybridized above the background level. Plasmid DNA was isolated from cultures of these colonies and digested with the restriction enzyme Sac I to check insert size. Six colonies exhibited a single insert and six showed multiple inserts. Four colonies with unique restriction patterns were sequenced using M13R and M13F universal sequencing primers homologous to the pUC19 vector. The M13F sequence of Clone 1, which had a single insert of approximately 3.9 Kb, showed homology to known phytoene desaturases. The remainder of this clone was sequenced by primer walking.

Homologies found for genes of interest are described in more detail in the BLAST Analyses section below. The three ORFs that showed homology to the lctA, lctB, and lctC genes of mediolanus were called lctA, lctB, and lctC genes of M. lueus ATCC 383.

Genome walidg was conducted to obtain the sequence of the C50-carotenoid operon upstream of the phytoene desaturase fragment. Genome walk libraries were made according to the protocol described for CLONTech's Universal Genome Walking Kit (CLONTech Laboratories, Inc., Palo Alto, Calif.). The restriction enzymes Hinc II, Stu I and Pvu II were used in making these libraries. The following primers were used in the procedure:

| | |
|---|---|
| GSP1F: | |
| 5′-TTCATGGACGTGCCCAGCAGCGTTGCCA-3′ | (SEQ ID NO: 42) |
| GSP2F: | |
| 5′-AGGTGGGCGAAGTCCGTGTAGAGGAAG-3′ | (SEQ ID NO: 43) |

GSP1F and GSP2F are primers facing upstream and GSP2F is nested inside of GSP1F. The addition of 5% DMSO to the PCR mixture was found to be necessary for amplification. First round PCR was conducted in a Perkin Elmer 9700 Thermocycler with 7 cycles consisting of 2 sec at 94° C. and 3 min at 72° C. and 34 cycles consisting of 2 sec at 94° C., and 3 min at 66° C., with a final extension at 66° C. for 4 min. Second round PCR used 5 cycles consisting of 2 sec at 94° C. and 3 min at 72° C. and 24 cycles consisting of 2 sec at 94° C. and 3 min at 66° C., with a final extension at 66° C. for 4 min. Nine μL of the first round product and seven liL of the second round product were run on a 1.5% TAE-agarose gel. A 0.9 Kb band was obtained for the second round product for the Hinc II library. This fragment was gel purified using a QIAquick Gel Purification Kit. Four μL of the purified DNA was ligated into pCR®II-TOPO vector and transformed by a heat-shock method into TOP10 E. coli cells using a TOPO cloning procedure (nvitrogen, Carlsbad, Calif.). Transformations were plated on LB media containing 100 μg/hL of ampicillin and 50 μg/mL of X-gal.

Individual, white colonies were screened by PCR using the GSP2F and AP2 primers. Individual colonies were resuspended in approximately 27 μl of 10 mM Tris and 2 μL of the resuspension was plated on LBK media (50 μg/mL kanamycin). The remnant resuspension was heated for 10 minutes at 95° C. to lyse the bacterial cells, and 2 μL of the heated cells used in a 25 μL PCR reaction. The PCR mix contained the following: 1× Taq buffer, 0.2 μM each primer, 0.2 mM each dNTP, 5% DMSO (v/v), and 1 unit of Taq polymerase per reaction. The PCR reaction was performed in a Perkin Elmer 9700 Thermocycler using the same program as used in the second round of genome walking. PCR product was separated on a 1% TAE-agarose gel along with remnant second round Hinc II product. Plasmid DNA for two colonies having inserts of the desired size was sequenced with the AP2 and GSP2F primers. The sequence obtained showed homology to known phytoene desaturases.

A second round of genome walking was conducted to obtain the remainder of the C50-carotenoid producing operon. The following primers were designed from the forward end of the sequence obtained from the first round of genome walking:

| | |
|---|---|
| GSP1F2: | |
| 5′-AAGTAGGTGCGTCCGAGCTGGTCGTGGT-3′ | (SEQ ID NO: 44) |
| GSP2F2: | |
| 5′-GTCCGCGCCGAGATCCCGCAGGAAGTT-3′ | (SEQ ID NO: 45) |

GSP1F2 and GSP2F2 are primers facing upstream and GSP2F2 is nested inside of GSP1F2.

These primers were used in PCR as described above and in the Genome Walker manual. A band of approximately 2.6 Kb was obtained for the second round PCR reaction using the Pvu II library. This DNA was gel purified, ligated into pCR®II-TOPO vector, and transformed into TOP10 E. coli cells using a TOPO cloning procedure. Individual colonies were screened by PCR for insert size, as previously described, using the AP2 and GSP2F2 primers. Plasmid DNA was obtained for a colony exhibiting an insert of the desired size and was sequenced using the GSP2F2 and AP2 primers. The remaining sequence for the insert was obtained by primer walking. PCR products for several regions of the operon were also sequenced to confirm the DNA sequence.

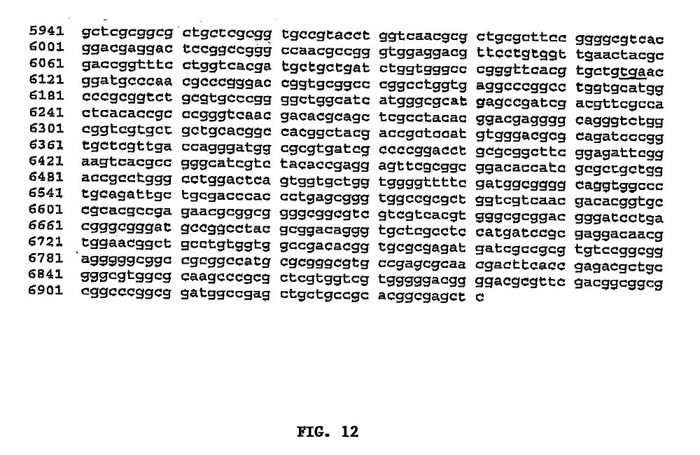

The full sequence of the operon, obtained by colony hybridization and genome waling, is given in FIG. 12.

As seen in FIG. 12, the operon isolated from M. lueus ATCC 383 comprises the following genes in order of location in the operon:
crtE, geranylgeranyl pyrophosphate synthase.
crtB, phytoene synthase.
crtI, phytoene dehydrogenase (phytoene desaturase).
lctA of M. lueus ATCC 383-having homology with lctA of A. mediolanus.
lctB of M. lueus ATCC 383-having homology with lctB of A. mediolanus.
lctC of M. lueus ATCC 383-having homology with lctC of A. mediolanus.
   C. Confirmation of C50 Production in M. lueus ATCC 383

C50 carotenoid (decaprenoxanthin) was produced in E. coli when the crtE-lctC gene fragment from M. lueus was cloned into E. coli together with the idi gene from E. coli on a pUC19 plasmid.

A gene construct containing the crtE, crtB, CrtI, lctA, lctB and lctC genes were inserted into the expression vector pProLarNde as described above. The idi gene from E. coli was cloned into the vector pUC19. These two plasmids were co-transformed into E. coli DH10B electrocompenet cells. Approximately 60 ng of the idi+pUC19 construct and 240 ng of crtE-lctC+pPRONde construct were used to electroporate 40 μL of ElectroMAX DH10BTM competent cells. Electroporated cells were recovered in SOC media for one hour and plated on LB plates containing 50 μg/ml of kanamycin, and 50 μg/ml of carbenicillin. Colonies were obtained after incubation at 37° C. and plated on LB plates containing 50 μg/ml of kanamycin, and 50 μ/ml of carbenicillin 1 mM IPTG, and 2% L-arabinose (LBKCIA) to induce gene expression from both vectors. After incubation colonies were scraped off the plate and extracted by the DMSO method of An et al. Cells were washed once with distilled water and once with acetone. The pellets were dried in air and resuspended in one ml of DMSO preheated to 55° C. Glass beads were added to each tube and vortexed to resuspend the pellets. One ml of acetone was added to extract the carotenoid, and one ml of hexane and two mls of 20% sodium chloride solution were added and the tubes vortexed. The phases were separated by centrifugation and the hexane phase was removed for carotenoid analysis. Spectrophotometric analysis between 350 and 500 nm revealed that the carotenoid profile matched that expected for decaprenoxanthin. These hexane carotenoid extracts were also subjected to mass spectrometer analysis and the expected Mass ion of 705.3 was observed in the E. coli double transformant as well as two additional mass ions at 687.4 and 669.6 corresponding the loss of one and two water molecules respectively. This mass of 705 (M+H) matches that expected for decaprenoxanthin.

D. BLAST Analyses to Determine Homology between Genes

BLAST searches of the above DNA sequence for M. lueus ATCC 383 against the Swisspro database identified the probable translational start and stop codons for the genes in the C50-carotenoid operon. The geranylgeranyl pyrophosphate (GGPP) synthase gene (crtE) for M. lueus ATCC 383 showed highest homology to the GGPP synthase gene of Brevibacterium linens (33% identity). The M. lueus ATCC 383 phytoene synthase gene (crtB) had highest homology to the phytoene synthase gene of Corynebacterium glutamicum (31% identity), followed by that of Brevibacterium linens. The phytoene desaturase gene (crt)) of M. lueus ATCC 383 showed highest homology to phytoene desaturase/dehydrogenase genes in Brevibacterium linens, Corynebacterium glutamicum, Halobacterium salinarium NRC-1, and Methanobacter thermautotrophicus, in order of decreasing homology.

The only significant BLAST hit for the M. lueus ATCC 383 lctA and lctB genes were to epsilon cyclase genes in Corynebacterium glutamicum (crtYe and crtyf, respectively, of Krubasik et al., Eur. J. Biochem. 268: 3702-3708 (2001)). The lctC gene of M. lueus ATCC 383 showed homology to lycopene elongase (crtEb of Krubasik et al.) from Corynebacterium glutamicum, followed by ORFs in Deinococcus radiodurans and Halobacterium salinarium NRC-1.

Alignments of Genes from M. lueus, A. mediolanus, and C. glutamicum)

Alignments for the crtE (GGPP synthesis genes), crtB (phytoene synthase genes), crtI (phytoene desaturase gene), lctA, crtYe, lctB, crtYf, lctC, and crtEb genes from M. luteus (M1), A. mediolanus (Am), and C. glutamicum (Cg) were aligned. Alignments were done using Align Plus software (Scientific and Educational Software, Durham, N.C.). These alignments were done using the multiway protein alignment fimction in conjunction with the BLOSUIM 62 matrix.

Results indicate that there is significant sequence identity shared between the amino acid sequences. These results indicate that the sequences could be used as substitutes for each other when they are used to create biosynthetic routes for generating C40, C45, and/or C50 carotenoids. Tables 3-8 provide a summary of the results from the alignments.

TABLE 3

| Gene | Start | End | Length | Matches | % Sequence Identity |
|---|---|---|---|---|---|
| M1- | 1 | 366 | 366 aa | 188 | 49% (M1-crtE and Am-crtE) |
| crtE | | | | | |
| Am- | 1 | 369 | 369 aa | 207 | 54% (Am-crtE and Cg-crtE) |
| crtE | | | | | |
| Cg- | 1 | 382 | 382 aa | 158 | 40% (Cg-crtE and MI-crtE) |
| crtE | | | | | |

TABLE 4

| Gene | Start | End | Length | Matches | % Sequence Identity |
|---|---|---|---|---|---|
| Mi- | 1 | 331 | 331 aa | 190 | 56% (MI-crtB and Am-crtB) |
| crtB | | | | | |
| Am- | 1 | 303 | 303 aa | 178 | 56% (Am-crtB and Cg-crtB) |
| crtB | | | | | |
| Cg- | 1 | 304 | 304 aa | 304 | 47% Cg-crtB and MI-crtB) |
| crtB | | | | | |

TABLE 5

| Gene | Start | End | Length | Matches | % Sequence Identity |
|---|---|---|---|---|---|
| Mi- | 1 | 543 | 543 aa | 337 | 59% (MI-crtI and Am-crtI) |
| crtI | | | | | |
| Am- | 1 | 544 | 544 aa | 364 | 65% (Am-crtI and Cg-crtI) |
| crtI | | | | | |
| Cg- | 1 | 549 | 549 aa | 308 | 54% (Cg-crtI and MI-crtI) |
| crtI | | | | | |

TABLE 6

| Gene | Start | End | Length | Matches | % Sequence Identity |
|---|---|---|---|---|---|
| Mi- | 1 | 115 | 115 aa | 62 | 52% (MI-lctA and Am-lctA) |
| lctA | | | | | |
| Am- | 1 | 123 | 123 aa | 67 | 45% (Am-lctA and Cg-crtYe) |
| lctA | | | | | |
| Cg- | 1 | 132 | 132 aa | 62 | 48% (Cg-crtYe and MI-lctA) |
| crtYe | | | | | |

TABLE 7

| Gene | Start | End | Length | Matches | % Sequence Identity |
|---|---|---|---|---|---|
| Mi- | 1 | 164 | 164 aa | 69 | 44% (MI-lctB and Am-lctB) |
| lctB | | | | | |
| Am- | 1 | 115 | 115 aa | 66 | 36% (Am-lctB and Cg-crtYf) |
| lctB | | | | | |
| Cg- | 1 | 130 | 130 aa | 53 | 42% (Cg-crtYf and MI-lctB) |
| crtYf | | | | | |

TABLE 8

| Gene | Start | End | Length | Matches | % Sequence Identity |
|---|---|---|---|---|---|
| Mi- | 1 | 291 | 291 aa | 206 | 66% (MI-lctC and Am-lctC) |
| lctC | | | | | |
| Am- | 1 | 298 | 298 aa | 199 | 57% (Am-lctC and Cg-crtEb) |
| lctC | | | | | |
| Cg- | 1 | 287 | 287 aa | 166 | 70% (Cg-crtEb and MI-lctC) |
| crtEb | | | | | |

### V. CONCLUSIONS

The experiments described above allowed for the isolation of the following seven (7) genes involved in the biosynthesis of the C50 carotenoid decaprenoxanthin in A. mediolanus:
isopentenyl pyrophosphate (diphosphate) isomerase (idi),
geranylgeranyl pyrophosphate synthase (crtE),
phytoene synthase (crtB),
phytoene desaturase (crtI),
lycopene ε-cyclase transferase A (lctA),
lycopene ε-cyclase transferase B (lctB), and
lycopene ε-cyclase transferase C (lctC).

Similar genes with substantial homology to the A. mediolanus genes were then isolated from M. lueus. It is believed that other similar genes with substantial homology could be isolated using similar techniques, and that such genes fall within the present invention.

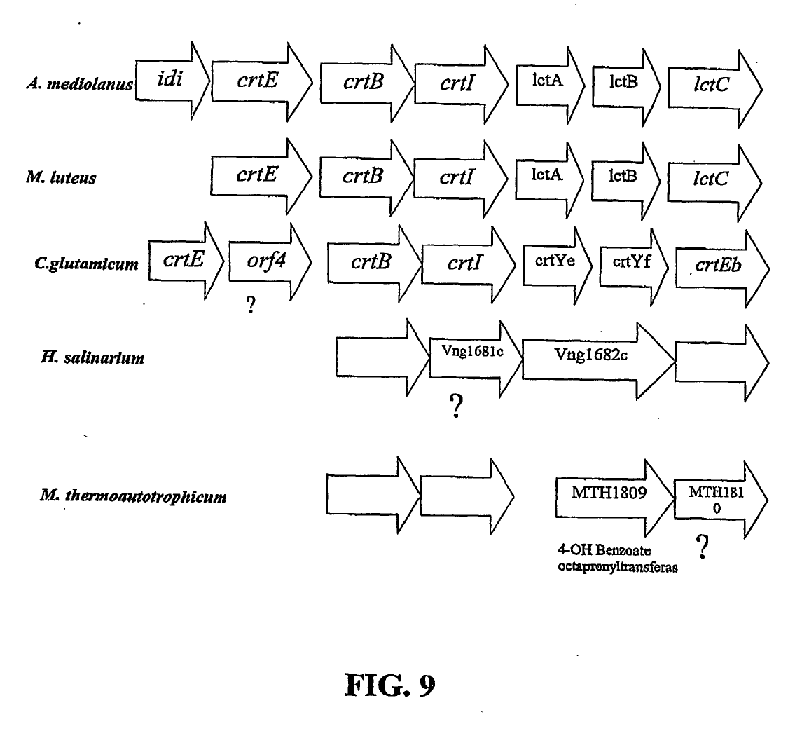

The experiments also show that there is a conservation in the gene arrangement between ORFs X1, X2 and Y, or Ict A, B and C genes respectively. A schematic comparison of the Ict A, B and C genes from,A. mediolanus and M. lueus with certain genes from other bacteria is shown in FIG. 9.

A schematic biosynthetic pathway, which is believed to summarize reactions of the present invention, is shown in FIG. 10. As has been shown, the Ict genes code for enzymes that react with the C40 carotenoid lycopene to perform two successive ε-cyclizations—coupled to the addition of C5 residues at the 2 and 2′ positions of the resulting carotenoid—to form (successively) a C45 (dehydrogenans-P452) and a C50 (decaprenoxanthin) carotenoid.

The invention provides genes capable of converting a C40 carotenoid to a C50 carotenoid. These genes (lctA, lctB, and lctC) are the first example of a set of genes that covert at C40 carotenoid to a C50 carotenoid in a single step. The three separate proteins can be used to convert a C40 carotenoid to the C50 carotenoid in a single step.

Some alternate uses of the genes described in this report are listed below. Some or all of the identified genes involved in lycopene biosynthesis (crtE, crtB, crtI) could be used alone, or in combination with carotenogenic genes from other organisms, in order to produce carotenoids such as (but not limited to): lycopene, β-carotene, lutein, zeaxanthin, canthaxanthin or astaxanthin. The gene for isopentenyl pyrophosphate isomerase (idi) could be utilized to increase the concentration of any carotenoids produced by a microorganism. This idi gene could be used in a genetic background that includes none, some or all of the other A. mediolanus carotenoid biosynthetic genes described here. A gene for carotenoid glycosyl transferase (e.g., zeaxanthin glycosyl transferase (crtX)) in a genetic background capable of producing dehydrogenans P-452, may be used to produce dehydrogenans P-452 monoglucoside; or (in a decaprenoxanthin producing background) to produce corynexanthin (decaprenoxanthin monoglucoside) or corynexanthin monoglucoside. Use of a carotenoid desaturase gene that is capable of adding additional conjugated double bonds to the C50 substrate will increase the antioxidant capacity of the molecule and change the spectral properties of the molecule (i.e. increasing the ₘₐₓ of the carotenoid). As mentioned before, sequence similarity searches of the Genbank public databases show three genes which have certain levels of homology to lctC. These genes are from carotenogenic organisms (Deinococcus radiodurans, Halobacterium sp. NRC-1, and Methanobacterium thermoautotrophicum) but their functions had not been previously defined. Because of the level of similarity between the gene sequences, it is probable that these three genes define a family of genes, all of which are involved in the conversion of C40 carotenoids to C>40 carotenoids. The Ict genes may be manipulated to perform other, related functions. These may include (but are not limited to): addition of the C5 residue without the associated cyclization reaction and/or addition of the C5 residue with a β-cyclization reaction (as opposed to the current ε-cyclization).

It is not difficult—through the use of additional enzymes like the FGPP synthase, combined with the genes isolated from A. mediolanus—to generate a fully conjugated novel C50 carotenoid with greatly improved antioxidant potential as well as unique absorption maxima. Such a molecule would result in carotenoids with novel colors. Similarly, modified phytoene desaturases-created by shuffling or by using other mutagenic techniques-could be employed with concepts of the present invention to create additional high performance carotenoids.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated polypeptide comprising at least one amino acid sequence selected from the group consisting of:
(a) the amino acid sequence set forth in SEQ ID NOS: 04, 05, 06, 10, 11, 12, 17, 18, 19, 20, 24, 25 or 26;
(b) an amino acid sequence having at least 10 contiguous amino acid residues of the amino acid sequence set forth in SEQ ID NOS: 04, 05, 06, 10, 11, 12, 17, 18, 19, 20, 24, 25 or 26;
(c) an amino acid sequence having one or more conservative amino acid substitutions within the amino acid sequence set forth in SEQ ID NOS: 04, 05, 06, 10, 11, 12, 17, 18, 19, 20, 24, 25 or 26; and
(d) an amino acid sequence having at least 65% sequence identity with the amino acid sequences of (a) or (b).

2. An isolated nucleic acid molecule encoding said polypeptide of claim 1.

3. The nucleic acid molecule of claim 2, wherein said polypeptide is capable of converting a C40 carotenoid to a C50 carotenoid.

4. The nucleic acid molecule of claim 2, wherein said polypeptide is capable of converting a C40 carotenoid to a C45 carotenoid.

5. The nucleic acid molecule of claim 2, wherein said polypeptide is capable of converting a C45 carotenoid to a C50 carotenoid.

6. The polypeptide of claim 1, wherein said polypeptide is capable of synthesizing a C40 carotenoid.

7. A production cell comprising said nucleic acid molecule of claim 2.

8. An isolated nucleic acid molecule comprising a nucleic acid sequence selected from the group consisting of:
(a) the nucleotide sequence set forth in SEQ ID NOS: 01, 02, 03, 07, 08, 09, 13, 14, 15, 16, 21, 22 or 23;
(b) a nucleic acid sequence having at least 10 contiguous nucleotides of the nucleotide sequence set forth in SEQ ID NOS: 01, 02, 03, 07, 08, 09, 13, 14, 15, 16, 21, 22 or 23;
(c) a nucleic acid sequence that hybridizes under moderately stringent conditions to the nucleotide sequence of (a); and
(d) a nucleic acid sequence having 65% sequence identity with the nucleic acid sequence of (a) or (b).

9. A production cell comprising said nucleic acid molecule of claim 8.

10. A method for making a C50 carotenoid, said method comprising contacting at least one of said polypeptides of claim 1 with a C40 carotenoid such that said CSO carotenoid is made.

11. A method for making a C50 carotenoid, said method comprising culturing said production cell of claim 7 under conditions wherein said C50 carotenoid is made.

12. A method for making a C45 carotenoid, said method comprising contacting at least one said polypeptide of claim 1 with a C40 carotenoid such that said C45 carotenoid is made.

13. A method for making a C45 carotenoid, said method comprising culturing the production cell of claim 7 under conditions wherein said C45 carotenoid is made.

14. A method for making a polypeptide, said method comprising culturing said production cell of claim 7 under conditions such that said polypeptide is made.

15. A specific binding agent that binds to said polypeptide of claim 1.

16. A method for making a C>40 carotenoid, said method comprising culturing a production cell, wherein said production cell comprises an exogenous nucleic acid molecule, wherein said exogenous nucleic acid molecule encodes a polypeptide that elongates a C>40 carotenoid by at least one carbon atom, wherein the product produced by said polypeptide is a carotenoid having a carbon backbone of >40 carbon atoms.

17. The method of claim 16, wherein said exogenous nucleic acid molecule comprises a nucleic acid sequence selected from the group consisting of:
(a) the nucleotide sequence set forth in SEQ ID NOS: 01, 02, 03, 07, 08, 09, 13, 14, 15, 16, 21, 22 or 23;
(b) a nucleotide sequence having at least 10 consecutive nucleotides of the nucleotide sequence set forth in SEQ ID NOS: 01, 02, 03, 07, 08, 09, 13, 14, 15, 16, 21, 22 or 23;
(c) a nucleic acid sequence that hybridizes under moderately stringent conditions to the nucleotide sequence of (a); and
(d) a nucleic acid sequence having 65% sequence identity with the nucleic acid sequence of (a) or (b).

18. The method of claim 16, wherein said exogenous nucleic acid molecule encodes a polypeptide, said polypeptide comprising at least one amino acid sequence selected from the group consisting of:
(a) the amino acid sequence of SEQ ID NOS: 04, 05, 06, 10, 11, 12, 17, 18, 19, 20, 24, 25 or 26;
(b) an amino acid sequence having at least 10 contiguous amino acid residues of the amino acid sequence set forth in SEQ ID NOS: 04, 05, 06, 10, 11, 12, 17, 18, 19, 20, 24, 25 or 26;
(c) an amino acid sequence having one or more conservative amino acid substitutions within the amino acid sequence of SEQ ID NOS: 04, 05, 06, 10, 11, 12, 17, 18, 19, 20, 24, 25 or 26; and
(d) an amino acid sequence having at least 65% sequence identity with the amino acid sequences of (a) or (b).


SEQUENCE LISTING (49 sequences)

SEQ ID NO: 1 - DNA, 372 bp, Agromyces mediolanus
  CDS (1)...(369)
atgaccttcctccacctggggctgctgctcgcctcgatcgcgtgcatcgcgctcgtcgacgcgcgctaccggctgttcttctggcgggcgccgctgcgggcgacggtcgtggtcgccctcggcgtcgcgatgctcctcgtctgggacctctggggcatctcgctcggcatcttcttccgcgagccgaatgcctactcgacggggctgctcattgcgccgcacctgccgatcgaggagccggtgttcctcgccttcctctgccagctcgcgatggtcggctacacgggactgctgcgcctcctcgcgcaccgatccgcgcagcccgccaccggccccgctgccgactccaccgccgaaggggcccgccgatga

SEQ ID NO: 2 - DNA, 348 bp, Agromyces mediolanus
  CDS (1)...(345)
atgagctacgccgtgctctgcctcccgttcctcgccgtctcggcggtgctcgccgcgatcgcctggcgacgtgctccggccggtcacgcggccgcgctcgcgctcacggcgggcggcctcgtgctcctcaccgcggtgttcgactcgctgatgatcgccgcgggcctgttcgactacgccgacgcgcccctgctcggcccgcgcctcgggctcgccccgatcgaggacttcgcctacccgatcgccgcgctgctgctctgctccacggtctggacgctgctcgggcgagcggatgcctcggcggctcgtgaccggcccgcccgcgcgcccagaggagccgagcgatga

SEQ ID NO: 3 - DNA, 897 bp, Agromyces mediolanus
  CDS (1)...(894)
atgagcgccgtcggcgccgaggcatccggccagcgcctgctccccgcgctcttcaccgcatcgcgcccgctgagctggatcaacaccgccttcccgttcgcggccgcgtacctgctgaccgtgcgcgaggtcgacgtcgcgctcgtcgtcggcaccctgttcttcctcgtgccgtacaacctcgcgatgtacggcatcaacgacgtcttcgacttcgagtccgacgcgcggaatccgcgcaagggcggcgtcgagggggccctgctgccgcccgcccggcatcgcgcggtgctgatcgccgcggtggccctgacggtgccgttcgtcgtctggctcgtgctgctcggcggcccgtggtcgtgggcctggctcgcgctcagcctgttcgccgtggtggcgtactcggcgccgggcctcaggttcaaggagatcccggggcctgactccctcacctcgagcacgcacttcgtctcgcccgcctgctacgggctcgccctcgcgggggcgacggtgacgccgcagctcgtgctgctgctgctcgcgttcttcgtgtggggcgtcgcgagccacgccttcggcgcggtgcaggacgtcgtgcccgatcgcgaggccgggatcgggtcgatcgcgaccgcgctgggggcccgccgcacgacccggctcgcgatcggcctctggctgctcgcgggcgtgctgatgctcggcacgtcgtggccggggccgctcgccgcggtactcgccgtgccgtacctcgtcgcggcgtggccgtaccgctcggtgagcgacgccgagtcggcgcgcgcgaacggcggctggcgctggttcctcgcgatcaactacggcgtcggcttcgcggcgacgatgctgctgatctggtacgcgctgctcacggcctga

SEQ ID NO: 4 - protein, 123 aa, Agromyces mediolanus
MTFLHLGLLLASIACIALVDARYRLFFWRAPLRATVVVALGVAMLLVWDLWGISLGIFFREPNAYSTGLLIAPHLPIEEPVFLAFLCQLAMVGYTGLLRLLAHRSAQPATGPAADSTAEGARR

SEQ ID NO: 5 - protein, 115 aa, Agromyces mediolanus
MSYAVLCLPFLAVSAVLAAIAWRRAPAGHAAALALTAGGLVLLTAVFDSLMIAAGLFDYADAPLLGPRLGLAPIEDFAYPIAALLLCSTVWTLLGRADASAARDRPARAPRGAER

SEQ ID NO: 6 - protein, 298 aa, Agromyces mediolanus
MSAVGAEASGQRLLPALFTASRPLSWINTAFPFAAAYLLTVREVDVALVVGTLFFLVPYNLAMYGINDVFDFESDARNPRKGGVEGALLPPARHRAVLIAAVALTVPFVVWLVLLGGPWSWAWLALSLFAVVAYSAPGLRFKEIPGPDSLTSSTHFVSPACYGLALAGATVTPQLVLLLLAFFVWGVASHAFGAVQDVVPDREAGIGSIATALGARRTTRLAIGLWLLAGVLMLGTSWPGPLAAVLAVPYLVAAWPYRSVSDAESARANGGWRWFLAINYGVGFAATMLLIWYALLTA

SEQ ID NO: 7 - DNA, 348 bp, Micrococcus luteus
  CDS (1)...(345)
atgtacctgctcctgctgctcgtcctcctgggctgtttcgcgctcatcgaccggcgctggaacctgtacttctggtccggacacccgctgcgggcctggctcgtgctggtcaccggggtggtgttcttcctcgcgtgggacctggtggggatcgccaacggactgttctggcacggcgagaactccctgaccctggggatcttcgtggctcccgagctgcccctggaagaggtcttcttcctcgcgttcctctgctaccagaccatggtctacgtgctcggcgcgcccgtgctgtggcggtggctgagggcccgcaccggcgcggcacacgcggggaggcgggcatga

SEQ ID NO: 8 - DNA, 495 bp, Micrococcus luteus
  CDS (1)...(492)
atgacgtactggggcgtgaacgcggtcttcctggggatggcggcggtcgtgctgctgacgacggcgctcgtgcggcgcccacccgcccggttctggggagcgctcgcggcctccacagtgctgctcgtggtgctcaccgccgtcttcgacaacgtcatgatcgcctccgggatcatgacgtacacggaccgcaacatctcgggcgtgcggatcgggctcgccccgctggaggacttcgcctaccccgtggccggtgtgctgctgctgccgacgatgtggctgctgctgggaggcacgcccggggcggcggccggtgacgggcgggcgacggcggcgtcgtcgtcctccgcggtcgcagccgcaaccgcagccggcgcgggcgacgagaacgcgagcggtgaggacgcggacaccgatggtacgagcaccgggcgcgcacatgccgggggcaggcccagtgggaaccccgccgatggaagggacgaaccgtgctga

SEQ ID NO: 9 - DNA, 876 bp, Micrococcus luteus
  CDS (1)...(873)
gtgctgaggacgctgttctgggcctcgcgcccgctgagctgggtgaacaccgcctacccgttcgcggcggccgtgctgctgacgggcggtttgccctggtggctcgtggcgctgggggccgtgttcttcctggtgccctacaacctggcgatgtacggcatcaacgacgtcttcgactacgagtcggacctgcgcaacccccgcaagggcggcgtggagggcgcggtggtggatcgcgccgcccagcgcggcgtgctgcgggcctcgtgcctgctgccggtgccgttcgtcgcggtgctggcggggtacgggatcgtgaccgggaacctgctgtccgtgctggtgctggcggtgagcctgttcgcggtggtcgcgtactcgtgggcggggctgcgctttaaggagcgcccgttcgtggatgcgatgacctccgccacccacttcgtctcgcccgccgtctacggactggtgctcgcacgggcggacttcacggtggggctgtgggcggtgctcgtgggcttcttcctgtggggcatggcctcgcagatgttcggggcggtgcaggacgtggtaccggaccgtgagggtgggctggcctccgtggccaccgtgctcggtgcgcgccccaccgtgtggctcgcggcgggcctctacgccctcgcaggtgccctgatgctgctcgcccagtggccgggtcagctcgcggcgctgctcgcggtgccgtacctggtcaacgcgctgcgcttccggggcgtcacggacgaggactccggccgggccaacgccgggtggaggacgttcctgtggttgaactacgcgaccggtttcctggtcacgatgctgctgatctggtgggcccgggttcacgtgctgtga

SEQ ID NO: 10 - protein, 115 aa, Micrococcus luteus
MYLLLLLVLLGCFALIDRRWNLYFWSGHPLRAWLVLVTGVVFFLAWDLVGIANGLFWHGENSLTLGIFVAPELPLEEVFFLAFLCYQTMVYVLGAPVLWRWLRARTGAAHAGRRA

SEQ ID NO: 11 - protein, 164 aa, Micrococcus luteus
MTYWGVNAVFLGMAAVVLLTTALVRRPPARFWGALAASTVLLVVLTAVFDNVMIASGIMTYTDRNISGVRIGLAPLEDFAYPVAGVLLLPTMWLLLGGTPGAAAGDGRATAASSSSAVAAATAAGAGDENASGEDADTDGTSTGRAHAGGRPSGNPADGRDEPC

SEQ ID NO: 12 - protein, 291 aa, Micrococcus luteus
VLRTLFWASRPLSWVNTAYPFAAAVLLTGGLPWWLVALGAVFFLVPYNLAMYGINDVFDYESDLRNPRKGGVEGAVVDRAAQRGVLRASCLLPVPFVAVLAGYGIVTGNLLSVLVLAVSLFAVVAYSWAGLRFKERPFVDAMTSATHFVSPAVYGLVLARADFTVGLWAVLVGFFLWGMASQMFGAVQDVVPDREGGLASVATVLGARPTVWLAAGLYALAGALMLLAQWPGQLAALLAVPYLVNALRFRGVTDEDSGRANAGWRTFLWLNYATGFLVTMLLIWWARVHVL

SEQ ID NO: 13 - DNA, 621 bp, Agromyces mediolanus
  CDS (1)...(618)
atgaccgacctcagcatcacgccgctgccggcccaggccgcaccggtgcagcccgcatccagcgccgaattggtcgtgctgctcgacgaggccggcaaccagatcggcaccgccccgaagtcgagcgtgcacggcgccgacaccgccctccatctcgcgttctcctgccacgtcttcgacgacgacggccgcctcctggtgacccgtcgcgcgctcggcaaggtcgcctggcccggcgtgtggaccaactccttctgcgggcaccccgccccggccgagccgctgccgcacgcggtgcgccgccgggccgagttcgagctcggcctcgagctccgcgacgtcgagccggtgctgccgttcttccgctaccgggcgacggatgcctcgggcatcgtcgagcacgagatctgcccggtctacacggcgcgcacaagctcggtgccggcgccgcatcccgacgaggtcctcgacctcgcctgggtcgaaccgggcgagctcgccaccgcggtccgcgccgcgccctgggcgttcagtccctggctcgtgctgcaggcgcagctgctgcccttcctcggcggccacgccgacgcgcgcgtccgcacggaagcgctcgtctcgtga

SEQ ID NO: 14 - DNA, 1110 bp, Agromyces mediolanus
  CDS (1)...(1107)
gtgagcctcgtcgcgaccgtggtcgccccgagccggcaggcggaggtggagcgctacctcggcggcttcttcgacgacgccatcgtgcgggccgacgcgcacgccgccgactaccggcggctctgggcggcggcgcgggacgccgcgagcggcggcaagcggatccgccccaggctcgtgctgggcgcctacgacgcgctcgccgcgcagggtgcgccggcgagcggccgcgaacgggccgacgccgagccggccgccgccgcggaggccgtggcgctcgcggcggccttcgagctgctgcacaccgcgttcctcgtgcacgacgacgtcatcgaccgcgacctcgtgcgccggggcgagcccaacgtcgccggccgcttcgcgctcgacgccgcgctgcgcgggctcgagcgggagcgggcggacgcctacggccaggcctcggcgatcctcgcgggcgacctgctgatcgcggcggcgcactccgtggcggccgcctcgacgtgccggtcgagcgccggcgagccatcctcgccgtccttgacgaagtgcgtcttcgccgccgccgcgggcgagcacgccgacgtccggcacgccgccggggtgcggcccggggaggcggacatcctcgcgatgatcgaggacaagacggcctgctactcgttcagcgcgccgctccgggcgggcgcgctgctcgccggcgccccgcgcgcgacggtcgaacggctcggcgagatcggccgtcgactcggcgtcgccttccagctgcaggacgacgtgctcggcgtctacggcgacgagcgggtgaccggcaagacggcgctcggggacctccgcgagggcaaggagacgctgctcatcgcctacgcgcgggggcacgcggcctgggtcgcggcatccggcgccttcggccggcccgacctcgacgaggcgggcgcccgccccctccgcgcggcgatcgaggcgagcggcgcccgcgcccgcgtcgaggcgcgcatcgccgaggaggcggccgcggcgcgcacggcgatcgccgcggcgggcctgcccgccgcgctcgaagccgagttgctcggcctcgccgccgaagccaccaggaggtcgaggtga

SEQ ID NO: 15 - DNA, 912 bp, Agromyces mediolanus
  CDS (1)...(909)
gtgagcacgcgcaccacccagcgcacgaccgcgccgcccgcaccgtccaccggcctcgccctctacgaccgcaccgccgccgagggctcggcccgggtcatccgggcgtactcgacctccttcggcctcgcgagccggctctgctcccccgccgtccgcgagcacctcgccgaggtctacgcgctcgtgcgcatcgccgacgagctcgtcgacggcccggccgaggaggccgggctgccgtgcgagcgccgccgcgagctgctcgacgccctcgaggccgacacggaggccgccttcgagagcggctacagcgccaacctcgtggtgcacgccttcgcgcgcgcggcgcggcgcagcggcttcggccaggagctcacccggcccttcttcgcctcgatgcgacgcgacctcgagcccatcgccttcaccgaggagcgcgagctcgacgaatacgtctacggctcggccgaggtcgtcggcctgatgtgcctgcgcggcttcgcgatcgggctcgcccccgacgccgagcgcgacgcccgctgggagcgcggcgcgcgggcgctgggctcggcgttccagcgggtcaacttcctgcgggacctcggggaggatgcctcgctccgcggacgccgctacttcccgggcgtcgatccggtgagcttctcggaggcccagcaactgcgcctcctcgacggcatcgacgcggagctcgacgaggcggccgccgtgatcccggagctgccccgcggctgccgcgtcgcggtcgccgcggcgcacggcctgttcggcgagctctccgcccggctccgccgcacgcccgcggccgagctcgtcacccggcgggtccgggtgcccgcgccgcgcaagctcgccatcgtcacccgcgtggtcgcccgcggaggccggccgtga

SEQ ID NO: 16 - DNA, 1635 bp, Agromyces mediolanus
  CDS (1)...(1632)
gtgagccgcgcggtcgtcatcggcggcggcatcgccgggctcgccacggcggcgctgctcgcccgcgacgggcacgaggtgcggctcttcgaggcgcgcgacgagctcggcggccgtgccgggcgctggcgggcgaacggcttcctgttcgacaccggtccgagctggtacctcatgccagaggtgttcgagcacttctaccgcttgatgggcaccacggcggccgaggagctcgagctcgtgcgcctcgaccccggctaccgggtgtacttcgagggctacgacgagccggtcgacgtgcgggccgagcgcgaggcatccatcgccctcttcgagtcgatcgagccgggcgcgggcgccgcgctcgcccggcacctcgactccgccaacgagacgtaccggctcgcgatgacgcacttcctctacaccgacttcgcccacccgggggcgctgctcgccgcgccggtccggcggcggctcggccggctcgcgaagctgctgctcgaaccgctcgaccgcatggtggggcgctccttcgacgacgtgcggctgcggcagatcctgggctacccggcggtcttcctcggcacctcgcccgagcgggcgccgagcatgtaccacctgatgagccgcttcgacctcgccgacggggtgttctacccgatgggcggcttcggcgagatcatcgcgagcgtggcccggctggcccggcgggccggggccgagctcgtcaccggcgcgcgggtgctcggcatcgagacggccggcgggcgcgccacgggcgtgcgcgtgcagcaccacggcccgaccggtggcaccggcaccgaggagttcctggaggccgagctcgtcgtctccgccgccgatctgcaccacacggatgccgagctgctcccgccccgcgcgcggacgcggagcgaggcatcctggtcgcgccgcgaccccggacccggcacggtgctcgtcatgctcggcgtgcacgggcggctgccggagctcgcccaccacacgctctgcttcacggccgactggcgcacgaacttccagcgggtgttcggctcgcgaccggcgatccccgacccggcgtcgttctacgtctgccgcccgagtgcgacggatccgggcgtggcgccccccggctgcgagaacctgttcctgctcgtgccggtgcccgccgaccccacaatcggcgccggcggtgtcgacggccgcggcgaccgggcggtcgaggagacggccgaccgggcgatcgcgaccctcgccgagtgggccggcatccccgacctcgccgagcggatcctcgtgcgccgcacgatcgggcccgcggacttcgaggactggttccagtcctggcgcggctcggcgctcggcccggggcacaccctgcggcagagcgccatgttccgggggcgcacggcctcggcgaacgtcgaggggctgtacttcgcgggggcgacgacgatcccgggcatcggcctgccgatgtgcctgatcagcgccgagctcgtcgcgaaggccgtgcgcggcgaggatgccccgggcccgctcccggagccgagcgaggagccgcacccagacccgctgcacccagacccgctgcacccagaccggctcgaccgggagcgcaccggatga

SEQ ID NO: 17 - protein, 206 aa, Agromyces mediolanus
MTDLSITPLPAQAAPVQPASSAELVVLLDEAGNQIGTAPKSSVHGADTALHLAFSCHVFDDDGRLLVTRRALGKVAWPGVWTNSFCGHPAPAEPLPHAVRRRAEFELGLELRDVEPVLPFFRYRATDASGIVEHEICPVYTARTSSVPAPHPDEVLDLAWVEPGELATAVRAAPWAFSPWLVLQAQLLPFLGGHADARVRTEALVS

SEQ ID NO: 18 - protein, 369 aa, Agromyces mediolanus
VSLVATVVAPSRQAEVERYLGGFFDDAIVRADAHAADYRRLWAAARDAASGGKRIRPRLVLGAYDALAAQGAPASGRERADAEPAAAAEAVALAAAFELLHTAFLVHDDVIDRDLVRRGEPNVAGRFALDAALRGLERERADAYGQASAILAGDLLIAAAHSVAAASTCRSSAGEPSSPSLTKCVFAAAAGEHADVRHAAGVRPGEADILAMIEDKTACYSFSAPLRAGALLAGAPRATVERLGEIGRRLGVAFQLQDDVLGVYGDERVTGKTALGDLREGKETLLIAYARGHAAWVAASGAFGRPDLDEAGARPLRAAIEASGARARVEARIAEEAAAARTAIAAAGLPAALEAELLGLAAEATRRSR

SEQ ID NO: 19 - protein, 303 aa, Agromyces mediolanus
VSTRTTQRTTAPPAPSTGLALYDRTAAEGSARVIRAYSTSFGLASRLCSPAVREHLAEVYALVRIADELVDGPAEEAGLPCERRRELLDALEADTEAAFESGYSANLVVHAFARAARRSGFGQELTRPFFASMRRDLEPIAFTEERELDEYVYGSAEVVGLMCLRGFAIGLAPDAERDARWERGARALGSAFQRVNFLRDLGEDASLRGRRYFPGVDPVSFSEAQQLRLLDGIDAELDEAAAVIPELPRGCRVAVAAAHGLFGELSARLRRTPAAELVTRRVRVPAPRKLAIVTRVVARGGRP

SEQ ID NO: 20 - protein, 544 aa, Agromyces mediolanus
VSRAVVIGGGIAGLATAALLARDGHEVRLFEARDELGGRAGRWRANGFLFDTGPSWYLMPEVFEHFYRLMGTTAAEELELVRLDPGYRVYFEGYDEPVDVRAEREASIALFESIEPGAGAALARHLDSANETYRLAMTHFLYTDFAHPGALLAAPVRRRLGRLAKLLLEPLDRMVGRSFDDVRLRQILGYPAVFLGTSPERAPSMYHLMSRFDLADGVFYPMGGFGEIIASVARLARRAGAELVTGARVLGIETAGGRATGVRVQHHGPTGGTGTEEFLEAELVVSAADLHHTDAELLPPRARTRSEASWSRRDPGPGTVLVMLGVHGRLPELAHHTLCFTADWRTNFQRVFGSRPAIPDPASFYVCRPSATDPGVAPPGCENLFLLVPVPADPTIGAGGVDGRGDRAVEETADRAIATLAEWAGIPDLAERILVRRTIGPADFEDWFQSWRGSALGPGHTLRQSAMFRGRTASANVEGLYFAGATTIPGIGLPMCLISAELVAKAVRGEDAPGPLPEPSEEPHPDPLHPDPLHPDRLDRERTG

SEQ ID NO: 21 - DNA, 1101 bp, Micrococcus luteus
  CDS (1)...(1098)
atgacctcggagacagacaccgcggcggatcccaccgcggtctgggatgtgttccgcgcggccgttgaccgggagctggacgagttcttcgactccccgcgcaacagggttccctacagcccgggcttcccggtgatgtgggatcgcatccggcagcaggtggtgggcggcaagctgatccggccccgtctgacgcagatcgcgtggcgctcgttcgccggtgagtcgagcactgactccggccgagaggccgagtgcgtgcgcctggcggcgtcgttcgagatgctgcacgcggcgctgatcgtgcacgacgacgtcgtggaccgggactggcgccgtcgtgggcggcccacggtgggcgagctcttccgccgcgacgcggtgcaggcgggggcccccgagggcgaggccgagcacgcgggggagtccgcggcgatcctcgcgggagacctgcttctggcgggtgcgctgcggctggcgaccacgtgcaccgaggacccggggcggggacgtgccgtggcagacgtggtcttcgaggcggtgaccgcgtccgcggccggtgagctggacgacctcctgctctctctgcaccgctacggcgcggagcacccgggcgtgcaggacatcctggacatggagcggctgaagaccgccacgtactcgttcgaggcacccctgcgcgccggcgccctgctcgcgggagcgcccgaggagcaggcccagcgcctggcgcgggccggcgcccagctcggggtggcctaccaggtcgtcgacgacgtcctgggaaccttcggcgaccccgagctcaccggcaagtcggtggacgccgatctgaactcgggcaaggccaccgtgctcaccgcccacggaatgcagacccccgcggtgcgggacgtcctcgcggagctcgcggccgggcgtaccacggtcgcctccgcgcgggctgccctgacggcgtcgggagcgcaggaggcagccgtggcagtggccacggacctcgtggaccgggcccgggccaccctggacggtctcccgctgcccgctgcccagcgcgcggagctcgacgcgctgtgccaccacgtcctgaacagagactcgtag

SEQ ID NO: 22 - DNA, 996 bp, Micrococcus luteus
  CDS (1)...(993)
gtgaggacccccaccatgccccaggacgcaccggccgacgcgccgctgagcctctacaccgccaccgcgctggcggcctcgggcgcggtgatcgggcgctactccacgtccttctcgctggcgtgccggaccctgccggcggcggtgcgccgggacatcgcggggatctacgccctcgtgcgcgtggcggacgaggtggtggacgggacggccggggcggcgggtctcggcgcggaccgggtgcgcgcggcgctcgacgcgtacgaggccgaggtggcctccgcgctcgccacgggcttctcgaccgacctggtggtccacggcttcgcgggcgtcgcccgccgtcacggcttcggcacggagctcacggagccgttcttcgcgtccatgcgcgcggacctggacgtggccgagcacgacggcgcctcgcttgagtcctacatctacggctcggcggaggtcgtggggctgatgtgcctggaggtcttcatggacatgcccggcacccgcgcccagaccccggagcagcgggagatgctgcgcgccacggcccgccggctgggtgccgcgttccagaaggtcaacttcctgcgggatctcggcgcggaccacgaccagctcggacgcacctacttccccggcgcggacccctcccacctggacgagacccgcaagcggctgctgctcgcggacctcggcgcggacctggacgcggccgtgcccgggatcctcgcgctggaccgccgtgccgggcgcgcggtgctgatcgcgcacggactgttcggtgagctcgcacggcggatcgaggaggtgcccgcggcggagctcacacgacggcgcatcagcgtgcccgccggggtgaagctgcggatcgccgcgagagcgctgtccgtcaccgcgcgcacgggctcacacgggcggggccgagccctagagtcggggcccccggtgccggcggccgtgcccgaaacctcccggacgggggccacccgatga

SEQ ID NO: 23 - DNA, 1632 bp, Micrococcus luteus
  CDS (1)...(1629)
atgacgcgcacggtggtgatcggcggcggcttcgcgggcctggccacggcgggcctgctcgcccgggacgggcacagcgtcaccctgctcgagcagcaggacacggtgggcggccgctccgggcggtggtccgcggagggcttctcgttcgacaccggacccagctggtacctcatgcccgaggtgatcgaccgctggttcaccctgatgggcacgagcgccgccgagcagctggacctgcgccggctggacccgggctaccgcgtcttcttcgaggaccacctggcggaaccgcccacggacgtggtcaccggtcgtgccgaggagctgttcgagagcctcgacccgggatcctcccgcgcactgcgctcctacctggactcgggcgcgcaggtctacgagctcgccaagaagcacttcctctacacggacttcgcccacctgctggaccttgtgcgcccggaggtgctccgcaacctcccgcggttggcaacgctgctgggcacgtccatgaagaactacgttgcgcgccgttttccggagccgcggcagcgccagatcctgggctaccccgccgtcttcctgggggcgtccccctcgtccgccccggccatgtaccacctcatgagccacctggacctcaccgacggagtgcagtacccggtgggcgggttcgccgcgctggtggacgccatggaacggctcgtgcgcgaggccggcgtggagatcgtcacgggagccaccgtgaccggcatcgaggtggctcccgagccgcggtcgccgcgttcccggttggccgcagcccgggcacgacgtcgcaccgccggcacggtcacgggcgtcaccttccgcacggcgccgggggcggacccggggacggagccgggcggcgtcgtcgccggtgcggaggtcaccgtgcccgcggacgtcgtcgtcggcgccgcggacctgcaccacctccagacccgcctgcttcccggcccgttccgcgcaccggagtcccgctggaagcgccgcgaccccgggccctccggggtgctcgtgtgcctgggcgtgcgcgggaagctgccgcagctggcccaccacaacctgctgttcaccgcggactgggatgagaacttcgggcgcatcgagtccggtgcggacctggccgaggagacctcgatctacgtgtccatgacgtcggcgacggatcccggcaccgcgcccgagggggacgagaacctgttcatcctggtgccctcgcccgcggcacccgagtggggtcacggcggaaccaccgccccgggcgtcgacgagcccggctccgcgcaggtggagcgggtcgctgacgccgccatcgcgcagctcgcgcgctgggcgcagatcccggacctggcctcgcggatcgtggtgcgcaggacctacgggcccgaggacttcgcggtgggggtcaacgcgtggcgcggctccctgctgggccccggacacattctgacgcagtccgcgatgttccgtcccagcgtcaccgaccgtgggatccgggggctgttctacgccgggtcctcggtgcgcccggggatcggcgtgcccatgtgcctgatctcctccgaggtggtgcgggacgccgtgcgggagagcggggcgcgctga

SEQ ID NO: 24 - protein, 366 aa, Micrococcus luteus
MTSETDTAADPTAVWDVFRAAVDRELDEFFDSPRNRVPYSPGFPVMWDRIRQQVVGGKLIRPRLTQIAWRSFAGESSTDSGREAECVRLAASFEMLHAALIVHDDVVDRDWRRRGRPTVGELFRRDAVQAGAPEGEAEHAGESAAILAGDLLLAGALRLATTCTEDPGRGRAVADVVFEAVTASAAGELDDLLLSLHRYGAEHPGVQDILDMERLKTATYSFEAPLRAGALLAGAPEEQAQRLARAGAQLGVAYQVVDDVLGTFGDPELTGKSVDADLNSGKATVLTAHGMQTPAVRDVLAELAAGRTTVASARAALTASGAQEAAVAVATDLVDRARATLDGLPLPAAQRAELDALCHHVLNRDS

SEQ ID NO: 25 - protein, 331 aa, Micrococcus luteus
VRTPTMPQDAPADAPLSLYTATALAASGAVIGRYSTSFSLACRTLPAAVRRDIAGIYALVRVADEVVDGTAGAAGLGADRVRAALDAYEAEVASALATGFSTDLVVHGFAGVARRHGFGTELTEPFFASMRADLDVAEHDGASLESYIYGSAEVVGLMCLEVFMDMPGTRAQTPEQREMLRATARRLGAAFQKVNFLRDLGADHDQLGRTYFPGADPSHLDETRKRLLLADLGADLDAAVPGILALDRRAGRAVLIAHGLFGELARRIEEVPAAELTRRRISVPAGVKLRIAARALSVTARTGSHGRGRALESGPPVPAAVPETSRTGATR

SEQ ID NO: 26 - protein, 543 aa, Micrococcus luteus
MTRTVVIGGGFAGLATAGLLARDGHSVTLLEQQDTVGGRSGRWSAEGFSFDTGPSWYLMPEVIDRWFTLMGTSAAEQLDLRRLDPGYRVFFEDHLAEPPTDVVTGRAEELFESLDPGSSRALRSYLDSGAQVYELAKKHFLYTDFAHLLDLVRPEVLRNLPRLATLLGTSMKNYVARRFPEPRQRQILGYPAVFLGASPSSAPAMYHLMSHLDLTDGVQYPVGGFAALVDAMERLVREAGVEIVTGATVTGIEVAPEPRSPRSRLAAARARRRTAGTVTGVTFRTAPGADPGTEPGGVVAGAEVTVPADVVVGAADLHHLQTRLLPGPFRAPESRWKRRDPGPSGVLVCLGVRGKLPQLAHHNLLFTADWDENFGRIESGADLAEETSIYVSMTSATDPGTAPEGDENLFILVPSPAAPEWGHGGTTAPGVDEPGSAQVERVADAAIAQLARWAQIPDLASRIVVRRTYGPEDFAVGVNAWRGSLLGPGHILTQSAMFRPSVTDRGIRGLFYAGSSVRPGIGVPMCLISSEVVRDAVRESGAR

SEQ ID NO: 27 - DNA, 30 bp, Artificial Sequence
  primer
ttcatatgtcactagccaggcgagatatcc

SEQ ID NO: 28 - DNA, 29 bp, Artificial Sequence
  primer
gaaagcttaagaagatgccgagcgagatg

SEQ ID NO: 29 - DNA, 30 bp, Artificial Sequence
  primer
agaagctttgtacggcacgaggaagaacag

SEQ ID NO: 30 - DNA, 28 bp, Artificial Sequence
  primer
gaaagcttctccgtgacgagatcctgag

SEQ ID NO: 31 - DNA, 33 bp, Artificial Sequence
  primer
gtcttaattaactgctgctctgctccacggtct

SEQ ID NO: 32 - DNA, 30 bp, Artificial Sequence
  primer
tatctagacgctccgtgacgagatcctgag

SEQ ID NO: 33 - DNA, 30 bp, Artificial Sequence
  primer
taggcatgcaacgtcgaggggctgtacttc

SEQ ID NO: 34 - DNA, 35 bp, Artificial Sequence
  primer
gctcgtcgacgcgcgctagccggctgttcttctgg

SEQ ID NO: 35 - DNA, 35 bp, Artificial Sequence
  primer
ccagaagaacagccggctagcgcgcgtcgacgagc

SEQ ID NO: 36 - DNA, 43 bp, Artificial Sequence
  primer
ggaacgggaggcagagcaggctagctcatcggcgggcccttcg

SEQ ID NO: 37 - DNA, 39 bp, Artificial Sequence
  primer
gggcccgccgatgagctagcctgctctgcctcccgttcc

SEQ ID NO: 38 - DNA, 35 bp, Artificial Sequence
  primer
gtgttgatccagctagcgggcgcgatgcggtgaag

SEQ ID NO: 39 - DNA, 35 bp, Artificial Sequence
  primer
ttcaccgcatcgcgcccgctagctggatcaacacc

SEQ ID NO: 40 - DNA, 19 bp, Artificial Sequence
  primer
agaggagccgagcgatgag

SEQ ID NO: 41 - DNA, 20 bp, Artificial Sequence
  primer
cgtaccagatcagcagcatc

SEQ ID NO: 42 - DNA, 28 bp, Artificial Sequence
  primer
ttcatggacgtgcccagcagcgttgcca

SEQ ID NO: 43 - DNA, 27 bp, Artificial Sequence
  primer
aggtgggcgaagtccgtgtagaggaag

SEQ ID NO: 44 - DNA, 28 bp, Artificial Sequence
  primer
aagtaggtgcgtccgagctggtcgtggt

SEQ ID NO: 45 - DNA, 27 bp, Artificial Sequence
  primer
gtccgcgccgagatcccgcaggaagtt

SEQ ID NO: 46 - DNA, 20 bp, Artificial Sequence
  exemplary sequence
aggtcgtgtactgtcagtca

SEQ ID NO: 47 - DNA, 20 bp, Artificial Sequence
  exemplary sequence
acgtggtgaactgccagtga

SEQ ID NO: 48 - DNA, 8651 bp, Agromyces mediolanus
  misc_feature (1)...(8651): n = A,T,C or G
ggatcacgggcagctcgacgccgcgccgggcgagctcggcctcgagtgcggccttcagctcgcggttctgctggttgatcgggctgatgccgtcgaagtggcggtagtggtgggcgacctcttcgaggcgctcgtcggggatgccccggccgctcgtgacgttccgcaggaaggggatgacgtcgtcctgcccctcgggcccgccgaagccggccagcaggatcgcgtcgtaggcgacgggctcggtgacgtgctcggggcccgactgggcggcctcggtggcaccgggcacgcaggcgcccgaggcgcagtacgcctcggcggcgggggccggcttgcggccgcgggcggcctcgcgctcgggcgcggcggctccggtcgagcccaggttcgtcgcggccattactggagcacctccacgagctcggcggtcgagatccgtcgaccggtgtagaacgggacctcttcgcgcacgtgcatgcgggcgtcggtggcgcgcagctcgcgcatgaggtcgacgagctcggtgagctcgtcggactcgatgggcagcagccactcgtagtcgccgagggcgaaggcgctcacggtgtgggcgatcgcgccgcggaaggtggcgcccttgcggccgtggtcggcgagcatgcgggagcgctcggccgggtcgagcaggtaccagtcgtagctgcgcacgaaggggtagaccgtcagccagcccttgggctcgatgccgcgcaggaagcccggcacgtgcgccttgttgaactcggcgtcgcggtgcacgcccatggcgttccacgtcggcagcagcgcgcgcagcaggcggctgcgcttcagctcgcgcagcgcccactgcaggccctcggcggtggcgccgtgcagccagatcatgacgtcggcgtcggcgcggaggccggagacgtcgtagagcccgcgcaccgtgacgccctcgttctcgacgagcgcgatgacgccgtcgagttcggtcacgaagcgcggcacatcgcgcccgtcgaggtcatcggggcgcgcggggtcctttcggagcacggcgaagagcgtgtagccctcgggcgactgctcgggttcggacgcgtgacggagctcgtcggctgccccttcggcagcgggggaagacatacccccagtctccctctttcccccggaaggtccaaaagggaggcgtcggctccgccgaatggcgcgggaatccgcggacggctcagtcctgtccggtcgcggcgagcgcgtcgaggaagcggatgacgacctcgcgctcctccggcgcgagggccgccgcggcggcgaagcgtcgcgcgtgctgacggccgacggtctcgcgggcgtcgtgtcgggtgtgctcggtgacggcgatcgcgagggcgcggcggtcgctcgggtggggcgatcgggtgacgtggccgccgcgttcgagccggtcgaggagcttcgtggtggaggcgctggagatgccgaggtgctcggcgagcgcgccgggcgtcacgacgagcccctggttgcgtgcggcgatgaggaagcggatggcgcgcatgtcggtctggttgagctgcatgtagcgccgggatgcctcgctcatgcgctcggcggcggcgtgccagccgcgcagcgcctgcatgacgcgcacgacctggtcgacctcctcgtcggcgagtccgctgcggtcgacgagctcctcgtcgcggtcgacgatgcgcggatcgtgcatcgccgattccacgcgccggctgcgctcccgctcatctgccatgtcgagattctagccaagcgagacgaatctcgctaagctactcactagccaggcgagatattcgccgcagcgagggttcggatcgagcacctcgcgccggagttgtcgaaggagccgacatgaccgacctcagcatcacgccgctgccggcccaggccgcaccggtgcagcccgcatccagcgccgaattggtcgtgctgctcgacgaggccggcaaccagatcggcaccgccccgaagtcgagcgtgcacggcgccgacaccgccctccatctcgcgttctcctgccacgtcttcgacgacgacgnccgcctcctggtgacccgtcgcgcgctcggcaaggtcgcctggcccggcgtgtggaccaactccttctgcgggcaccccgccccggccgagccgctgccgcacgcggtgcgccgccgggccgagttcgagctcggcctcgagctccgcgacgtcgagccggtgctgccgttcttccgctaccgggcgacggatgcctcgggcatcgtcgagcacgagatctgcccggtctacacggcgcgcacaagctcggtgccggcgccgcatcccgacgaggtcctcgacctcgcctgggtcgaaccgggcgagctcgccaccgcggtccgcgccgcgccctgggcgttcagtccctggctcgtgctgcaggcgcagctgctgcccttcctcggcggccacgccgacgcgcgcgtccgcacggaagcgctcgtctcgtgagcctcgtcgcgaccgtggtcgccccgagccggcaggcggaggtggagcgctacctcggcggcttcttcgacgacgccatcgtgcgggccgacgcgcacgccgccgactaccggcggctctgggcggcggcgcgggacgccgcgagcggcggcaagcggatccgccccaggctcgtgctgggcgcctacgacgcgctcgccgcgcagggtgcgccggcgagcggccgcgaacgggccgacgccgagccggccgccgccgcggaggccgtggcgctcgcggcggccttcgagctgctgcacaccgcgttcctcgtgcacgacgacgtcatcgaccgcgacctcgtgcgccggggcgagcccaacgtcgccggccgcttcgcgctcgacgccgcgctgcgcgggctcgagcgggagcgggcggacgcctacggccaggcctcggcgatcctcgcgggcgacctgctgatcgcggcggcgcactccgtggcggccgcctcgacgtgccggtcgagcgccggcgagccatcctcgccgtccttgacgaagtgcgtcttcgccgccgccgcgggcgagcacgccgacgtccggcacgccgccggggtgcggcccggggaggcggacatcctcgcgatgatcgaggacaagacggcctgctactcgttcagcgcgccgctccgggcgggcgcgctgctcgccggcgccccgcgcgcgacggtcgaacggctcggcgagatcggccgtcgactcggcgtcgccttccagctgcaggacgacgtgctcggcgtctacggcgacgagcgggtgaccggcaagacggcgctcggggacctccgcgagggcaaggagacgctgctcatcgcctacgcgcgggggcacgcggcctgggtcgcggcatccggcgccttcggccggcccgacctcgacgaggcgggcgcccgccccctccgcgcggcgatcgaggcgagcggcgcccgcgcccgcgtcgaggcgcgcatcgccgaggaggcggccgcggcgcgcacggcgatcgccgcggcgggcctgcccgccgcgctcgaagccgagttgctcggcctcgccgccgaagccaccaggaggtcgaggtgaccgcgctcccgatcggcgctgcgttgctcggcctcgccgccgaagccaccaggaggaccaggtgagcacgcgcaccacccagcgcacgaccgcgccgcccgcaccgtccaccggcctcgccctctacgaccgcaccgccgccgagggctcggcccgggtcatccgggcgtactcgacctccttcggcctcgcgagccggctctgctcccccgccgtccgcgagcacctcgccgaggtctacgcgctcgtgcgcatcgccgacgagctcgtcgacggcccggccgaggaggccgggctgccgtgcgagcgccgccgcgagctgctcgacgccctcgaggccgacacggaggccgccttcgagagcggctacagcgccaacctcgtggtgcacgccttcgcgcgcgcggcgcggcgcagcggcttcggccaggagctcacccggcccttcttcgcctcgatgcgacgcgacctcgagcccatcgccttcaccgaggagcgcgagctcgacgaatacgtctacggctcggccgaggtcgtcggcctgatgtgcctgcgcggcttcgcgatcgggctcgcccccgacgccgagcgcgacgcccgctgggagcgcggcgcgcgggcgctgggctcggcgttccagcgggtcaacttcctgcgggacctcggggaggatgcctcgctccgcggacgccgctacttcccgggcgtcgatccggtgagcttctcggaggcccagcaactgcgcctcctcgacggcatcgacgcggagctcgacgaggcggccgccgtgatcccggagctgccccgcggctgccgcgtcgcggtcgccgcggcgcacggcctgttcggcgagctctccgcccggctccgccgcacgcccgcggccgagctcgtcacccggcgggtccgggtgcccgcgccgcgcaagctcgccatcgtcacccgcgtggtcgcccgcggaggccggccgtgagccgcgcggtcgtcatcggcggcggcatcgccgggctcgccacggcggcgctgctcgcccgcgacgggcacgaggtgcggctcttcgaggcgcgcgacgagctcggcggccgtgccgggcgctggcgggcgaacggcttcctgttcgacaccggtccgagctggtacctcatgccagaggtgttcgagcacttctaccgcttgatgggcaccacggcggccgaggagctcgagctcgtgcgcctcgaccccggctaccgggtgtacttcgagggctacgacgagccggtcgacgtgcgggccgagcgcgaggcatccatcgccctcttcgagtcgatcgagccgggcgcgggcgccgcgctcgcccggcacctcgactccgccaacgagacgtaccggctcgcgatgacgcacttcctctacaccgacttcgcccacccgggggcgctgctcgccgcgccggtccggcggcggctcggccggctcgcgaagctgctgctcgaaccgctcgaccgcatggtggggcgctccttcgacgacgtgcggctgcggcagatcctgggctacccggcggtcttcctcggcacctcgcccgagcgggcgccgagcatgtaccacctgatgagccgcttcgacctcgccgacggggtgttctacccgatgggcggcttcggcgagatcatcgcgagcgtggcccggctggcccggcgggccggggccgagctcgtcaccggcgcgcgggtgctcggcatcgagacggccggcgggcgcgccacgggcgtgcgcgtgcagcaccacggcccgaccggtggcaccggcaccgaggagttcctggaggccgagctcgtcgtctccgccgccgatctgcaccacacggatgccgagctgctcccgccccgcgcgcggacgcggagcgaggcatcctggtcgcgccgcgaccccggacccggcacggtgctcgtcatgctcggcgtgcacgggcggctgccggagctcgcccaccacacgctctgcttcacggccgactggcgcacgaacttccagcgggtgttcggctcgcgaccggcgatccccgacccggcgtcgttctacgtctgccgcccgagtgcgacggatccgggcgtggcgccccccggctgcgagaacctgttcctgctcgtgccggtgcccgccgaccccacaatcggcgccggcggtgtcgacggccgcggcgaccgggcggtcgaggagacggccgaccgggcgatcgcgaccctcgccgagtgggccggcatccccgacctcgccgagcggatcctcgtgcgccgcacgatcgggcccgcggacttcgaggactggttccagtcctggcgcggctcggcgctcggcccggggcacaccctgcggcagagcgccatgttccgggggcgcacggcctcggcgaacgtcgaggggctgtacttcgcgggggcgacgacgatcccgggcatcggcctgccgatgtgcctgatcagcgccgagctcgtcgcgaaggccgtgcgcggcgaggatgccccgggcccgctcccggagccgagcgaggagccgcacccagacccgctgcacccagacccgctgcacccagaccggctcgaccgggagcgcaccggatgaccttcctccacctggggctgctgctcgcctcgatcgcgtgcatcgcgctcgtcgacgcgcgctaccggctgttcttctggcgggcgccgctgcgggcgacggtcgtggtcgccctcggcgtcgcgatgctcctcgtctgggacctctggggcatctcgctcggcatcttcttccgcgagccgaatgcctactcgacggggctgctcattgcgccgcacctgccgatcgaggagccggtgttcctcgccttcctctgccagctcgcgatggtcggctacacgggactgctgcgcctcctcgcgcaccgatccgcgcagcccgccaccggccccgctgccgactccaccgccgaaggggcccgccgatgagctacgccgtgctctgcctcccgttcctcgccgtctcggcggtgctcgccgcgatcgcctggcgacgtgctccggccggtcacgcggccgcgctcgcgctcacggcgggcggcctcgtgctcctcaccgcggtgttcgactcgctgatgatcgccgcgggcctgttcgactacgccgacgcgcccctgctcggcccgcgcctcgggctcgccccgatcgaggacttcgcctacccgatcgccgcgctgctgctctgctccacggtctggacgctgctcgggcgagcggatgcctcggcggctcgtgaccggcccgcccgcgcgcccagaggagccgagcgatgagcgccgtcggcgccgaggcatccggccagcgcctgctccccgcgctcttcaccgcatcgcgcccgctgagctggatcaacaccgccttcccgttcgcggccgcgtacctgctgaccgtgcgcgaggtcgacgtcgcgctcgtcgtcggcaccctgttcttcctcgtgccgtacaacctcgcgatgtacggcatcaacgacgtcttcgacttcgagtccgacgcgcggaatccgcgcaagggcggcgtcgagggggccctgctgccgcccgcccggcatcgcgcggtgctgatcgccgcggtggccctgacggtgccgttcgtcgtctggctcgtgctgctcggcggcccgtggtcgtgggcctggctcgcgctcagcctgttcgccgtggtggcgtactcggcgccgggcctcaggttcaaggagatcccggggcctgactccctcacctcgagcacgcacttcgtctcgcccgcctgctacgggctcgccctcgcgggggcgacggtgacgccgcagctcgtgctgctgctgctcgcgttcttcgtgtggggcgtcgcgagccacgccttcggcgcggtgcaggacgtcgtgcccgatcgcgaggccgggatcgggtcgatcgcgaccgcgctgggggcccgccgcacgacccggctcgcgatcggcctctggctgctcgcgggcgtgctgatgctcggcacgtcgtggccggggccgctcgccgcggtactcgccgtgccgtacctcgtcgcggcgtggccgtaccgctcggtgagcgacgccgagtcggcgcgcgcgaacggcggctggcgctggttcctcgcgatcaactacggcgtcggcttcgcggcgacgatgctgctgatctggtacgcgctgctcacggcctgagccgtcgctccgcggngagggcgcgagtccgcgagcgcgtcactgcccgtcgaggggcgtcactccccgtcgaggggcggcgatccgagcaggagcccggtcgagtgggcgatgtgccgccgcatcgcctcgacgccctcggcctgcacctcggcgagcagctcgcggtgctcgacgacgagttcggcgaggctgtagtgccggcggatgtgcagcaggaagagccgcagctcggcgccgagcgcggcgtgcgcctcgacgatcctggggctgccgctggcggcgacgatcgcccggtgcacctcgaggtggagccgctcggcctcgagccaggccggggtcgtctcgcgctcgcccggaggacgcagcgcctcctcggtgacggcgagccgggcgagctcgtcgagggcgagcatcgccggggcgagcgccgcctcgggccagtgcgcgccgtagcggtcgcccgcgatgcgtaccgcttcgacctcgagcgcctcgcgcagttgctgcagcgcgagcacctgcgcgtggtcgaactcggtgacccgcactccgcggtagggcgccgactcggcgagccgctcagcgacgagccgctggaacgcggcgcgcacggtgtgccgggacaccccgaagcgctcggccgcctgctcctcgcgcagcggcgcccccgaggccagcgcgccgctcaggatctcgtcacggagcgcatcggccatccgctcgacggcggtgggcgccggcatgacgcggcggctcagtcgtcgctgacggcagcgcgcacgacgagggcgacgacgccggcgaccacgacgaccgccccgatcc

SEQ ID NO: 49 - DNA, 6941 bp, Micrococcus luteus
ctgcccccgctgctcgtgcacgccatccggttcggcggcggctacgggggtgcggtggtgcgggccctgcgccagctcgggtgaccccgcccgtggttggacaggacccgccgctgtccagcatgatggttattagaatttctagtagttacgaggcgggagtcaccgggtgacggagaccggagcgtggagtgcgagcgtgagcccgcagtcgcgcgcgctgcgtcggctggtgcggctgaacgaggggatcgggtaccagatccgccgcctcatgggcctgaaggaaaccgactactccgccatggccctgctcttgcggagtccgatggggcccaccgacctggcccacgctctgcacatcaccaccgcttccgccacggccgtggtggaccggctcgcacgggccggtcacgtggtgcgtgaaccgcacggagaggaccgccgccgcatgaccgtgcgggccgtggccggatcccgtgagcaggtgcgggagcacgtggtgcccatgatggacatggtcgaggaggagctcgcgcggctggacgagtccggccgcggggccgtcctgcagttcctcaccggcaccgccgaccgcatggaggactacctggcgggtctgcgcgaacgcccggccggcactggcggcgccacccagggcatgcccggccccggggcggagcgcccatgacctcggagacagacaccgcggcggatcccaccgcggtctgggatgtgttccgcgcggccgttgaccgggagctggacgagttcttcgactccccgcgcaacagggttccctacagcccgggcttcccggtgatgtgggatcgcatccggcagcaggtggtgggcggcaagctgatccggccccgtctgacgcagatcgcgtggcgctcgttcgccggtgagtcgagcactgactccggccgagaggccgagtgcgtgcgcctggcggcgtcgttcgagatgctgcacgcggcgctgatcgtgcacgacgacgtcgtggaccgggactggcgccgtcgtgggcggcccacggtgggcgagctcttccgccgcgacgcggtgcaggcgggggcccccgagggcgaggccgagcacgcgggggagtccgcggcgatcctcgcgggagacctgcttctggcgggtgcgctgcggctggcgaccacgtgcaccgaggacccggggcggggacgtgccgtggcagacgtggtcttcgaggcggtgaccgcgtccgcggccggtgagctggacgacctcctgctctctctgcaccgctacggcgcggagcacccgggcgtgcaggacatcctggacatggagcggctgaagaccgccacgtactcgttcgaggcacccctgcgcgccggcgccctgctcgcgggagcgcccgaggagcaggcccagcgcctggcgcgggccggcgcccagctcggggtggcctaccaggtcgtcgacgacgtcctgggaaccttcggcgaccccgagctcaccggcaagtcggtggacgccgatctgaactcgggcaaggccaccgtgctcaccgcccacggaatgcagacccccgcggtgcgggacgtcctcgcggagctcgcggccgggcgtaccacggtcgcctccgcgcgggctgccctgacggcgtcgggagcgcaggaggcagccgtggcagtggccacggacctcgtggaccgggcccgggccaccctggacggtctcccgctgcccgctgcccagcgcgcggagctcgacgcgctgtgccaccacgtcctgaacagagactcgtagtgaggacccccaccatgccccaggacgcaccggccgacgcgccgctgagcctctacaccgccaccgcgctggcggcctcgggcgcggtgatcgggcgctactccacgtccttctcgctggcgtgccggaccctgccggcggcggtgcgccgggacatcgcggggatctacgccctcgtgcgcgtggcggacgaggtggtggacgggacggccggggcggcgggtctcggcgcggaccgggtgcgcgcggcgctcgacgcgtacgaggccgaggtggcctccgcgctcgccacgggcttctcgaccgacctggtggtccacggcttcgcgggcgtcgcccgccgtcacggcttcggcacggagctcacggagccgttcttcgcgtccatgcgcgcggacctggacgtggccgagcacgacggcgcctcgcttgagtcctacatctacggctcggcggaggtcgtggggctgatgtgcctggaggtcttcatggacatgcccggcacccgcgcccagaccccggagcagcgggagatgctgcgcgccacggcccgccggctgggtgccgcgttccagaaggtcaacttcctgcgggatctcggcgcggaccacgaccagctcggacgcacctacttccccggcgcggacccctcccacctggacgagacccgcaagcggctgctgctcgcggacctcggcgcggacctggacgcggccgtgcccgggatcctcgcgctggaccgccgtgccgggcgcgcggtgctgatcgcgcacggactgttcggtgagctcgcacggcggatcgaggaggtgcccgcggcggagctcacacgacggcgcatcagcgtgcccgccggggtgaagctgcggatcgccgcgagagcgctgtccgtcaccgcgcgcacgggctcacacgggcggggccgagccctagagtcggggcccccggtgccggcggccgtgcccgaaacctcccggacgggggccacccgatgacgcgcacggtggtgatcggcggcggcttcgcgggcctggccacggcgggcctgctcgcccgggacgggcacagcgtcaccctgctcgagcagcaggacacggtgggcggccgctccgggcggtggtccgcggagggcttctcgttcgacaccggacccagctggtacctcatgcccgaggtgatcgaccgctggttcaccctgatgggcacgagcgccgccgagcagctggacctgcgccggctggacccgggctaccgcgtcttcttcgaggaccacctggcggaaccgcccacggacgtggtcaccggtcgtgccgaggagctgttcgagagcctcgacccgggatcctcccgcgcactgcgctcctacctggactcgggcgcgcaggtctacgagctcgccaagaagcacttcctctacacggacttcgcccacctgctggaccttgtgcgcccggaggtgctccgcaacctcccgcggttggcaacgctgctgggcacgtccatgaagaactacgttgcgcgccgttttccggagccgcggcagcgccagatcctgggctaccccgccgtcttcctgggggcgtccccctcgtccgccccggccatgtaccacctcatgagccacctggacctcaccgacggagtgcagtacccggtgggcgggttcgccgcgctggtggacgccatggaacggctcgtgcgcgaggccggcgtggagatcgtcacgggagccaccgtgaccggcatcgaggtggctcccgagccgcggtcgccgcgttcccggttggccgcagcccgggcacgacgtcgcaccgccggcacggtcacgggcgtcaccttccgcacggcgccgggggcggacccggggacggagccgggcggcgtcgtcgccggtgcggaggtcaccgtgcccgcggacgtcgtcgtcggcgccgcggacctgcaccacctccagacccgcctgcttcccggcccgttccgcgcaccggagtcccgctggaagcgccgcgaccccgggccctccggggtgctcgtgtgcctgggcgtgcgcgggaagctgccgcagctggcccaccacaacctgctgttcaccgcggactgggatgagaacttcgggcgcatcgagtccggtgcggacctggccgaggagacctcgatctacgtgtccatgacgtcggcgacggatcccggcaccgcgcccgagggggacgagaacctgttcatcctggtgccctcgcccgcggcacccgagtggggtcacggcggaaccaccgccccgggcgtcgacgagcccggctccgcgcaggtggagcgggtcgctgacgccgccatcgcgcagctcgcgcgctgggcgcagatcccggacctggcctcgcggatcgtggtgcgcaggacctacgggcccgaggacttcgcggtgggggtcaacgcgtggcgcggctccctgctgggccccggacacattctgacgcagtccgcgatgttccgtcccagcgtcaccgaccgtgggatccgggggctgttctacgccgggtcctcggtgcgcccggggatcggcgtgcccatgtgcctgatctcctccgaggtggtgcgggacgccgtgcgggagagcggggcgcgctgatgtacctgctcctgctgctcgtcctcctgggctgtttcgcgctcatcgaccggcgctggaacctgtacttctggtccggacacccgctgcgggcctggctcgtgctggtcaccggggtggtgttcttcctcgcgtgggacctggtggggatcgccaacggactgttctggcacggcgagaactccctgaccctggggatcttcgtggctcccgagctgcccctggaagaggtcttcttcctcgcgttcctctgctaccagaccatggtctacgtgctcggcgcgcccgtgctgtggcggtggctgagggcccgcaccggcgcggcacacgcggggaggcgggcatgacgtactggggcgtgaacgcggtcttcctggggatggcggcggtcgtgctgctgacgacggcgctcgtgcggcgcccacccgcccggttctggggagcgctcgcggcctccacagtgctgctcgtggtgctcaccgccgtcttcgacaacgtcatgatcgcctccgggatcatgacgtacacggaccgcaacatctcgggcgtgcggatcgggctcgccccgctggaggacttcgcctaccccgtggccggtgtgctgctgctgccgacgatgtggctgctgctgggaggcacgcccggggcggcggccggtgacgggcgggcgacggcggcgtcgtcgtcctccgcggtcgcagccgcaaccgcagccggcgcgggcgacgagaacgcgagcggtgaggacgcggacaccgatggtacgagcaccgggcgcgcacatgccgggggcaggcccagtgggaaccccgccgatggaagggacgaaccgtgctgaggacgctgttctgggcctcgcgcccgctgagctgggtgaacaccgcctacccgttcgcggcggccgtgctgctgacgggcggtttgccctggtggctcgtggcgctgggggccgtgttcttcctggtgccctacaacctggcgatgtacggcatcaacgacgtcttcgactacgagtcggacctgcgcaacccccgcaagggcggcgtggagggcgcggtggtggatcgcgccgcccagcgcggcgtgctgcgggcctcgtgcctgctgccggtgccgttcgtcgcggtgctggcggggtacgggatcgtgaccgggaacctgctgtccgtgctggtgctggcggtgagcctgttcgcggtggtcgcgtactcgtgggcggggctgcgctttaaggagcgcccgttcgtggatgcgatgacctccgccacccacttcgtctcgcccgccgtctacggactggtgctcgcacgggcggacttcacggtggggctgtgggcggtgctcgtgggcttcttcctgtggggcatggcctcgcagatgttcggggcggtgcaggacgtggtaccggaccgtgagggtgggctggcctccgtggccaccgtgctcggtgcgcgccccaccgtgtggctcgcggcgggcctctacgccctcgcaggtgccctgatgctgctcgcccagtggccgggtcagctcgcggcgctgctcgcggtgccgtacctggtcaacgcgctgcgcttccggggcgtcacggacgaggactccggccgggccaacgccgggtggaggacgttcctgtggttgaactacgcgaccggtttcctggtcacgatgctgctgatctggtgggcccgggttcacgtgctgtgaacggatgcccaacgcccgggaccggtgcggcccggcctggtgaggcccggcctggtgcatggcccgcggtctgcgtgcccggggctggcatcatgggcgcatgagccgatcgacgttcgccactcacaccgcccgggtcaacgacacgcagctcgcctacacggacgaggggcagggtctggcggtcgtgctgctgcacggccacggctacgaccgctccatgtgggacgcgcagatcccggtgctcgttgaccagggatggcgcgtgatcgccccggacctgcgcggcttcggagattcggaagtcacgccgggcatcgtctacaccgaggagttcgcggcggacaccatcgcgctgctggaccgcctgggcctggactcagtggtgctggtggggttttcgatggcggggcaggtggccctgcagattgctgcgacccaccctgagcgggtggccgcgctggtcgtcaacgacacggtgccgcacgccgagaacgcggcggggcggcgtcgtcgtcacgtgggcgcggacgggatcctgacgggcgggatgccggcctacgcggacagggtgctcgcctccatgatccgcgaggacaacgtggaacggctgcctgtggtggccgacacggtgcgcgagatgatcgccgcgtgtccggcggagggggcggccgcggccatgcgcgggcgtgccgagcgcaacgacttcaccgagacgctgcgggcgtggcgcaagcccgcgctcgtggtcgtgggggacggggacgcgttcgacggcggcgcggcccggcggatggccgagctgctgccgcacggcgagctc